(19) United States
(12) Patent Application Publication
(10) Pub. No.: US 2005/0050594 A1
(43) Pub. Date: Mar. 3, 2005

(54) Tryptophan synthase as a site of herbicide action

(76) Inventors: Shirley Rodaway, Ewing, NJ (US); Karl-Heinz Ott, Lawrenceville, NJ (US); Charles M. Langevine, Brooklyn, NY (US); Laura Sarokin, Skillman, NJ (US); Genichi Kakefuda, Princeton Junction, NJ (US); John Finn, Stow, MA (US)

Correspondence Address: James Remenick;Intellectual Property Department, Morrison & Foerster LLP, 1650 Tyson's Boulevard, Suite 300, McLean, VA, 22102, US

(21) Appl. No.: 10/818,451

(22) Filed: Apr. 6, 2004

Related U.S. Application Data
(63) Continuation of application No. 09/498,949, filed on Feb. 4, 2000.
(60) Provisional application No. 60/119,208, filed on Feb. 5, 1999.

(51) Int. Cl. A01H 5/00

(52) U.S. Cl. 800/300

(57) ABSTRACT

The invention relates to methods of identifying inhibitors of tryptophan synthase (TS) that are useful as herbicides, the TS inhibiting herbicides, methods of designing variants of the TS enzyme that are resistant to the herbicides of the invention and other known herbicides, the TS enzyme variants themselves, polynucleotides encoding these TS enzyme variants, plants expressing the TS enzyme variants, and methods of weed control.

### FIELD OF THE INVENTION

The invention relates to methods of identifying inhibitors of tryptophan synthase (TS) that are useful as herbicides, the TS inhibiting herbicides, methods of designing variants of the TS enzyme that are resistant to the herbicides of the invention and other known herbicides, the TS enzyme variants themselves, polynucleotides encoding these TS enzyme variants, plants expressing the TS enzyme variants, and methods of weed control.

### BACKGROUND OF THE INVENTION

There is an increasing need in agriculture for herbicides with novel mechanisms of action, compounds that are targeted to new processes, pathways, and enzymes in plants. Each individual herbicide may injure a different set of weeds. The spectrum of weeds in various crops is continuously changing, as climatic and edaphic factors change, and as ecological changes lead to less obvious weeds becoming more prolific. The latter is a consequence of both ongoing and new agricultural practices eliminating otherwise more competitive species from the agroecosystem. Thus new herbicide chemicals are of value. New herbicide targets are of even greater value since older herbicide targets can be compromised when natural variants in weed populations become more abundant on farms where older herbicides have been used for a long time. As a result, new herbicides with new modes of action are needed to address the following issues in agriculture: the development of shifting weed populations, the inadvertent selection of resistant weeds, and the need for specific agrochemicals with improved environmental characteristics. Moreover, with greater emphasis on transgenic crops with herbicide resistance traits, there is a need for not only novel chemistries but also for associated novel resistant herbicide target genes.

Applicants have now surprisingly discovered that TS, an enzyme involved in tryptophan biosynthesis, is a useful target site for herbicides. The lack of homologous genes of TS and the tryptophan synthesis pathway in animals is advantageous since the herbicides designed according to the present invention are not toxic for humans and animals.

Tryptophan synthase (TS) catalyzes the final two reactions in tryptophan biosynthesis and is composed of four subunits, two α subunits and two β subunits. The TSα subunit catalyzes a retroaldol reaction in which indoleglycerol-3-phosphate (IGP) is cleaved to yield indole and D-glyceraldehyde-3-phosphate (GAP). Indole from the TSα subunit reaction is channeled via a 25 angstrom tunnel to the β subunit active site. The β subunit catalyzes the condensation of L-serine and indole to form tryptophan. FIG. 1 shows these reactions. Tryptophan, which is synthesized in this reaction, is one of the essential amino acids. There is evidence that tryptophan is a precursor of the plant hormone, indole acetic acid.

Attempts have been made to identify inhibitors of TS. For example, the substrate analog, indole-3-propanol phosphate (IPP) was described as an inhibitor of TSα subunit (Kirschner et al., Eur. J. Biochem., 1975, 60:513). However, as shown in the Examples, the level of inhibition of the enzymatic activity by IPP is modest. The compound is without any herbicidal activity.

Shuto et al. (Pesticide Sci., 1989, 14:69) tested certain pyridine derivatives for their ability to inhibit TS, in an older assay thought to test for inhibition of the TSβ reaction. Shuto tested a few such compounds on rice plants and saw a reduction in plant growth only for one, 2-mercaptobenzimidazole (MBI). However, Shuto did not show whether TS was a direct target for MBI. The mechanism of action, ie., whether the reduction in growth resulted from the inhibition of tryptophan biosynthesis (as opposed to non-selective inhibition of many enzymes) is not evident from this article. Compounds were not shown to specifically interact with the TS enzyme complex nor were experiments done to investigate whether supplying exogenous tryptophan can reverse the injurious effect of the inhibitor. This compound, although the most active enzyme inhibitor described by Shuto is much less active against TS than IPP. Thus, even ten years after the publication of the Shuto article, there remains in the art, the need for direct inhibitors of TS which have herbicidal activity.

The present inventors have now experimentally proven that TS is a direct target for the inhibitors of the invention by using the herbicide-reversal method and crystallographic studies. They have therefore, surprisingly discovered the methods of the present invention (e.g. high throughput screening for TS inhibitors, structure-based design of TS inhibitors, and methods for development of herbicide resistance genes) and their use for identifying effective herbicides.

### SUMMARY OF THE INVENTION

The present invention relates to identifying herbicides that are TS inhibitors and that act by binding to TS and inhibiting tryptophan biosynthesis, the novel herbicides, and the methods of using these herbicides for weed control.

Accordingly, in one aspect of the invention, inhibitors of TS having the property of binding to TS and inhibiting tryptophan biosynthesis, as well as isolated complexes of TS and the inhibitor of the invention are provided.

In another embodiment, methods for identifying novel TS inhibitors using (i) a structure-based approach and/or (ii) targeted high throughput compound screening are provided.

In another aspect of the invention, methods of purifying plant TS from plant tissues or from bacterial cultures containing recombinantly produced plant TS and such purified plant enzymes are also provided.

In yet another aspect, the invention provides for variants of the TS enzyme that are resistant to inhibition by the inhibitors of the present invention, and transgenic crop plants expressing variant TS.

In a further aspect, the invention provides for methods of weed control using the herbicides identified according to the present invention.

### BRIEF DESCRIPTION OF THE DRAWINGS

FIG. 1 is a scheme showing the TSα subunit and TSβ subunit reactions.

FIG. 2 is a graph showing chemical structures of the phosphonate inhibitors 1 to 5 of tryptophan synthase.

FIGS. 3A-3E are schematic drawings of hydrogen bonding interactions and relative distances between the five phosphonate inhibitors and catalytic residues at the α subunit active site: (A) Inhibitor 1; (B) Inhibitor 2; (C) Inhibitor 3; (D) Inhibitor 4; and (E) Inhibitor 5.

FIG. 4 represents a complex of TS with indole-propanol-3-phosphonic acid (purple space filling model in the active site pocket of αTS, indicated by a wire-mesh diagram, outlining the Connolly surface (1.4 Å probe radius, colored by the Delphi-generated electrostatic potential.) Note: the poor filling of the pocket below the indole plane and the conformation of the inhibitor.

FIG. 5 represents a complex of TS with {4-[(2-amino-5-methoxy-phenyl)thio]butyl}-phosphonic acid in the pocket. Note the improved filling of the binding site, increasing the affinity by improved van-der-Waals contacts.

FIG. 6 represents a view of the binding site for the indole ring system in αTS. The yellow surface indicated the Connolly-surface of the αTS binding pocket. The blue, ball-and-stick model represents the position of the indole ring as found in the X-Ray structure (2trs). The red stick-model represents the position of {4-[(2-amino-5-methoxy-phenyl)thio]butyl}-phosphonic acid. Selected fragment hits from the LUDI search are represented by green lines. It is shown that the addition of a bulky group such as the methoxy group of {4-[(2-amino-5-methoxy-phenyl-thio]butyl}-phosphonic acid occupies part of the space. In fact, the X-Ray structure of this compound in complex with TS indicates that the Methoxy group undergoes extensive rotation consistent with this model.

FIG. 7 shows a Ludi Fragment hit #019 overlaid onto the structure of TS with {4-[(2-amino-5-methoxy-phenyl)thio]butyl}-phosphonic acid bound to the active site. It is evident that the program found a fragment with an OH replacing the NH group as an interaction site with αAsp60. While inhibitor binding is slightly reduced by replacement of NH2 by OH, the phenolic group results in a much better herbicidal profile, probably due to the increased acidity that results in increased uptake and translocation.

FIG. 8 shows superposition of indole-propanol-phosphate bound to TS and {4[(2-amino-5-methoxy-phenyl)thio]butyl}-phosphonic acid (Green sphere in center) extends into a pocket created by, between others, αA129 (space filled representation, left) and αIle 153 (space-filled model, right; these sites are highly attractive targets for mutations.

### DETAILED DESCRIPTION OF THE INVENTION

All patents, patent applications and references cited herein are hereby incorporated by reference in their entirety. In case of any inconsistency, the present disclosure governs.

The present invention relates to identifying herbicides that are TS inhibitors, the novel herbicides, crops genetically engineered to be resistant to these herbicides and the methods of using the herbicides for weed control.

Herbicidal Inhibitors

Herbicidal inhibitors of TS specifically listed herein as well as the inhibitors identified using the methods described below have the property of binding to TS and abrogating tryptophan synthesis. The herbicidal effect of these inhibitors can be shown to be prevented or substantially ameliorated by coordinately supplying tryptophan to the living organism or tissue. As used herein, the term “herbicidal inhibitor” means a compound that (i) binds to TS and has the property of inhibiting tryptophan synthesis (in vitro and/or in vivo) and (ii) is effective as a herbicide.

A compound is considered “effective as an inhibitor” if the concentration required to eliminate 50% of enzyme activity (I₅₀) is in the range from low nM to about 20 μM. In one embodiment, the I₅₀ value is a maximum of about 10 μM, preferably a maximum of about 1 μM and most preferably less. In another embodiment, the level of enzymatic activity is less than 500 nM.

A compound is considered “effective as a herbicide” if the plant or plant tissue dies or is severely damaged or stunted, such that it would no longer be expected to survive to produce seed, or to be agroecologically competitive after it has been treated with the compound. For a compound to be an effective herbicide, it must provide a means of injuring plants. The amount of compound required will depend on a number of factors, but one of the factors will be that the compound interferes with a critical process in the plant when used at a reasonable concentration of inhibitor. This concentration can be measured in vitro, and it stands to reason that, all other factors being equal, the compound that is most inhibitory in vitro has the potential to be the most inhibitory as a herbicide. Commercially viable herbicides will inhibit 50% of the activity of a target enzyme at concentrations below 20 μM and preferably below 1 μM.

As referred herein, “in vitro” means outside of a plant organism. The term includes both cell-free and cell-containing systems (e.g. assays).

The herbicidal inhibitors of the invention may bind to any active site of the enzyme, such as for example, the active site of a or β subunits or the hydrophobic tunnel connecting the subunits. In one embodiment of the invention, the herbicidal inhibitors of the invention are compounds that bind to the active site of the a subunit.

In a preferred embodiment of the invention, herbicidal TS inhibitors are arylthioalkyl- and arylthioalkenylphosphonic acids and derivatives having the structural formula I: 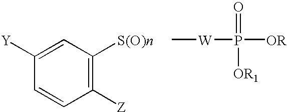
wherein
Y is hydrogen or halogen;
Z is NH₂or OR₂;
R₂ is hydrogen, C₁-C₄alkylcarbonyl or benzoyl;
n is an integer of 0, 1 or 2;
W is —(CH₂)₄—, —CH₂CH═CHCH₂— or —CH₂CH₂CH═CH—; and
R and R₁ are each independently hydrogen, C₁-C₄alkyl,
C₁-C₄ alkylcarbonyloxymethylene or an alkali metal,
ammonium or organic ammonium cation.

Preferred formula I herbicidal agents of the present invention are those wherein
Y is hydrogen, F or Br;
Z is NH₂ or OR₂;
R₂ is hydrogen, C₁-C₄ alkylcarbonyl or benzoyl;
n is an integer of 0 or 1;
W is —(CH₂)₄— or —CH₂CH₂CH═CH—; and
R and R₁ are each independently hydrogen, C₁-C₄alkyl, C₁-C₄alkylcarbonyloxymethylene or an alkali metal or organic ammonium cation.

Arylthioalkyl- and arylthioalkenylphosphonic acids and derivatives of the present invention which are particularly effective herbicidal agents include
{4-[(o-hydroxyphenyl)thio]-1-butenyl}phosphonic acid;
diethyl {4-[(o-aminophenyl)thio]butyl}phosphonate;
dilithium {4-[(o-aminophenyl)thio]butyl}phosphonate;
(4-[(o-aminophenyl)thio]butyl}phosphonic acid, compound with cyclohexylamine (1:2);
dipivalate ester of bis(hydroxylmethyl) {4-[(o-hydroxyphenyl) thio]butyl}phosphonate;
{4-[(o-hydroxyphenyl)sulfinyl]butyl}phosphonic acid, compound with cyclohexylamine (1:2);
(4-[(o-hydroxyphenyl)thio]butyl}phosphonic acid, compound with N,N,N¹N¹-tetra methyl ethylene diamine;
{4-[(o-hydroxyphenyl)sulfinyl]butyl}phosphonic acid;
4-[(o-hydroxyphenyl)thio]butenyl}phosphonic acid, arylbutyrate ester; and
{4-[(o-hydroxyphenyl)thio]-1-butenyl}phosphonic acid, compound with isopropylamine (1:2), among others.

Examples of halogen hereinabove are fluorine, chlorine, bromine and iodine. In formula I above, alkali metals may include: sodium, potassium and lithium. Further, the term organic ammonium is defined as a group consisting of one or two positively charged nitrogen atoms each joined to form one to four C₁-C₁₆alkyl groups, provided that when the group contains two positively charged nitrogen atoms, the organic ammonium cations R and R₁ are each present in the same group. These preferred herbicidal inhibitor of the invention may be prepared as described in the U.S. Pat. No. 5,635,449.

In addition to the herbicidal inhibitors described above, any herbicidal inhibitor described herein, or identified using methods described herein, is within the scope of the invention. In one embodiment, the herbicidal inhibitor is as described herein but is not the inhibitor of formula I.

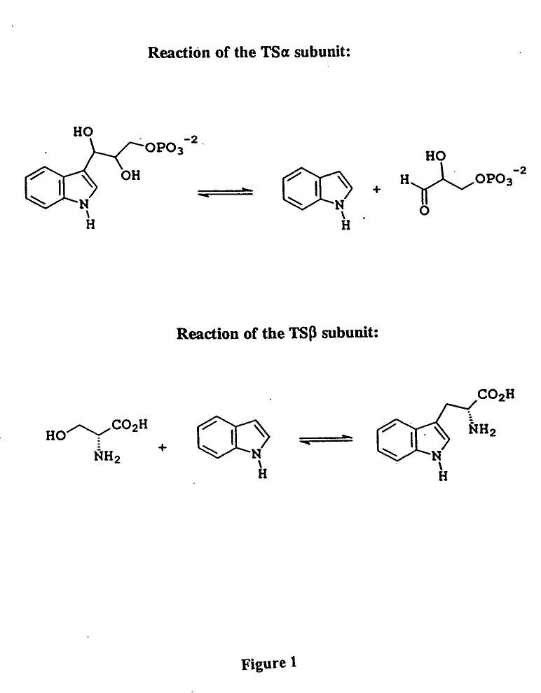

The herbicidal inhibitors of the invention that bind to the active site of the α subunit may mimic the structure of the natural TSα substrate, indole-3-glycerol phosphate (IGP) and its intermediate product (both represented in FIG. 1). Referring to FIG. 1, IGP and its reaction intermediate contain an indole ring, an alkyl chain linker and a phosphate.

In one embodiment, the herbicidal inhibitors differ from the original substrate IGP in at least one of the following aspects: (i) the C2 atom of the indole ring is removed resulting in a 6-member ring; (ii) the indol-NH group is replaced with a hydrogen bond donor having the property of interacting with the amino acid αD60 of the TSα subunit (NH, hydroxyl, or similar groups may be used); (iii) the linker region is constructed to be preferably hydrophobic, (iv) the linker may contain one or more C═C double bonds, (v) the linker has a length similar to the length of a linear chain of four single bonded carbon atoms (the linker is C₄H₈ similar) and (vi) the phosphate group is replaced with the phosphonate group. Substituents such as halogens, may be added to the 6-member ring, which can influence the electron density in the pi-electron cloud and affect the aromatic stacking and binding of the aromatic ring of the inhibitor. The linker may contain, in addition to a chain of methylene groups, amides, C═C double bonds, or even ring systems, like cyclohexyl, or phenyl groups. In one embodiment of the invention, the C3 atom may be replaced with sulfur (S) (e.g., FIG. 1).

All amino acids referred hereto are designated by their one-letter code and their position in the enzyme. The amino acid position numbers are in reference to the TS enzyme from Salmonella. The prefix “α” indicates that the aminoacid is located in the TSα subunit. The prefix β indicates that the amino acid is located in the TSβ subunit.

The herbicidal inhibitors of the invention may further be modified and tested using the methods of the present invention. For example, additional groups may be added to better fill the enzyme binding site or to interact with other groups that line the enzyme binding site. For example, additional polar groups could be added to the linker or, elsewhere in the vicinity of the indol C3 or sulfur position. This polar group, the additional hydrogen-bond donor on the linker such as an NH or hydroxyl group, can interact with the amino acids of TSα αY175-OH or αE49 to further improve the binding. Another modification may involve reshaping of the aromatic ring system to optimize placement of the hydrogen bond donor that interacts with αD60.

Further, modifications may be designed to improve the herbicidal activity of the inhibitors. Chemical modifications of charged or polar groups (such as the phosphate/phosphonate, or the hydroxy or amino groups) may be designed by additions of fragments that can be removed by chemical or enzymatic cleavage after application. These modifications may be designed to improve metabolic stability, uptake, and/or translocation. For example, the esterification or salt formation of an in vitro active inhibitor greatly increases its herbicidal activity. Similarly, reduction of the basicity of the anilino-group by replacing it with a phenol-OH group, and subsequent masking of that hydroxy group, leads to the currently most potent herbicides for TS. Similarly, other groups, like sulfonamides can be used to mask the amino or the phosphonate groups.

Based on the crystallographic studies of the TS enzymes with a bound inhibitor of the above formula I (some of which are described in Example 18), the interactions between the TS enzyme and its inhibitors have been discerned. Based on these interactions, some of which are described below, additional inhibitors may be designed and evaluated.

Polar interactions of the phosphonate group with the TS protein include a network of hydrogen bonds and electrostatic interactions. One of the phosphonate oxygen atoms interacts directly with the amide hydrogen of αG213 and αG184. The second phosphonate oxygen interacts with the backbone HN of αG234 and with a tightly bound water molecule, that further forms a hydrogen bond to the carbonyl group of α232. The water's oxygen interacts with the amide hydrogen atoms of αI214 and αF212. This water molecule is located in the extend of the axis of α-helix αK243 to αS235. This helix is designated Helix H8′ according to Hyde 1988 (Hyde et al., J. Biol. Chem. 263, 33 (1988) 17857), and is thought to contribute to the binding of the phosphate group through its dipolar field. In addition, the side chain functionality of helix H8′-terminating αS235 and its carbonyl group both interact strongly with the third phosphonate oxygen atom. As shown in the Example 18, the αS235/phosphonate interaction is through a very strong hydrogen bond. The present study has shown (as the electron density map contoured at 2sigma) continuous electron density between these two groups. Close to the αS235 hydroxyl group is another electron density spot that is attributed to a bound water molecule. Another positively charged group, the guanidinium group of αR179, is close to the phosphonate without undergoing direct hydrogen bonding interactions. Analysis of the electrostatic interaction surface (calculated using Finite Element Poisson-Bolzman calculations) created by the protein indicates a strongly positive potential where the phosphonate group is bound. This positive potential is created by the action of HN groups pointing toward the phosphonate and the presence of R199.

Replacements of the phosphate group with other charged groups was not well tolerated by the TS enzyme. This is likely because the phosphate is bound rather specifically by directional hydrogen bonds, contributed by the backbone amino acid groups rather than by a less directional salt interaction. However, for herbicide design purposes, groups that can be metabolized to yield a phosphate or phosphonate, such as esters and sulfonamides, are preferred for plant uptake purposes.

Furthermore, there are two additional distinct binding pockets adjacent to the phosphonate binding site. These sites may be filled by suitable ligands to improve binding affinity and selectivity. Those ligands may be designed by using fragment-based searches (for example, as described below using the LUDI program).

The aliphatic chain that connects the phosphonate with the aryl group is the linker region. It is bound to the enzyme channel that is wide enough to allow for a considerable flexibility. The electron density of {4-[2-amino-5-chlorophenyl)thio]butyl}phosphonic acid bound to the TS enzyme suggests rotational freedom for the dihedrals of the linker chain. The surface of the TS channel lining in contact with the linker is partially hydrophobic due to the side chains of αF22 and αI64. However, polar groups, such as αY175-OH and backbone amides lead to a partially polarized enzyme surface without necessarily providing direct hydrogen bonding contacts as for the glyceryl portion of the substrate. Introduction of hydrogen bond donors/acceptors, e.g. in the form of amide groups in the linker region did not lead to increased binding affinity, indicating that formation of a hydrogen bond does not compensate the entropy loss due to the introduction of a hydrophobic group inside a rather hydrophobic enzyme site. Increasing the rigidity of the linker region by means of a C═C double bond, on the other hand, does increase the free energy of binding.

LUDI searches conducted to design modifications of the linker region, suggest that there is enough space for introduction of a phenyl or cyclohexyl group, ie., molecules of the form aryl-S-cyclohexyl-phosphonate are also within the scope of the invention. Those modifications are not expected to greatly improve the binding affinity of the compound, but are suitable for introducing metabolic handles for improved herbicide selectivity or for improved uptake and translocation.

The thioaryl group of the inhibitors of the invention binds into the indol-binding pocket with the o-amino group pointing toward αD60. The thio-ester sulfur atom is located relatively deep in the hydrophobic pocket created by αF22, αI232, αL100, αL127, and αY175 when αE49 folds away from the presumable site of the enzymatic cleavage and forms water-mediated hydrogen bonds to αY4 and αS125. The binding of the thioaryl group is considerably different from the previously described binding of indol derivatives: the thioaryl ring is shifted and tilted relative to the position of the indole derivatives in complex with TS. The aromatic portion of the inhibitors is sandwiched between αL100 on and αF212. The plane of the phenyl groups of αF212 is orthogonal to the plane of the aryl group of the inhibitors. The T-shaped stacking of αF212 and the aryl group of the inhibitor/substrate is indicative of a t-shaped pi-pi interaction. (Burley, S. K. and Petsko, G. A., Science, 229, (1985) 23.)

The back bone of αF212 adopts a conformation φ/ψ=−75/155, that is considered “forbidden” energetically for free amino acids and is clearly justified by the electron density. This is in contrast to results from earlier X-ray analysis of the IGP/TS complex as reported by Rhee et al., J. Biol. Chem., 273:8553-5, 1998. The electron density of the Phosphonic acid, {4-[(2-amino-5-chlorophenyl)thio]butyl}-/TS complex also indicates increased electron density at CZ of αF212. This apparent change in the backbone position reveals a strong correlation between phosphonate binding and aryl group binding that has now been discovered based on the X-ray studies reported herein.

The relative position of αF212 and the thioaryl group to each other, (that was unknown prior to this work and could not have been derived by analogy from the IPP/TS complex studies) shows that the electron density at the individual atom position in the aryl group is very critical for the binding affinity. The loss of affinity in a pyridine analog, and the binding affinity for the series para-substituted thio-aryl analogs (substituent is para to the sulfur, meta to the amino group) substituted with R═Br>Cl>OMe>H>CH₃, is clearly explained by a T-shaped aryl-aryl stacking interaction in which the hydrogen atoms of αF212 bind into the π electron cloud of the thioaryl group. Increasing the electron delocalization at the para position to the sulfur is thereby expected to be critical for binding. Further, it is not necessary that the para-substituent is small, in fact, larger substituents will be well tolerated and can be used to gain herbicide selectivity since those substituents would extend into a region of the protein that is less strongly conserved among the species. Thus, groups of the type O—R, S—R, etc. are candidates for improved herbicides.

The amino group of the inhibitors is involved in a network of polar interactions that, first of all, include the salt bridge with the carboxylate functionality of αD60, which further interacts with αT183, αY102-OH, αN68-NH2, and a water molecule. The primary amino group is in the orientation forming bidentate hydrogen bonds with αD60. However, the corresponding H—O distances of 2.2 Å and 3.0 Å are rather long. The amino group is also in proximity to αF22 and could have a polarizing effect on this aromatic system. A hydroxy-group in place of the amino group has advantages in terms of herbicidal properties. This is attributed to the reduced basicity relative to the amino functionality. Thus, groups that mask the amino group, for example, sulfonamide derivatives will have an improved herbicidal profile.

Electrostatic potential calculations show that αG49 is protonated in the free enzyme as well as in the complex with inhibitors. This destabilizes the enzyme by about 10 kJ/mol. Introducing another basic group to interact with αG49 is expected to release this energy in the form of increased binding affinity. Hence, additions of, e.g. an amino group, in a suitable location, i.e., at the beginning of the linker region, is expected to be beneficial. However, steric requirements will need to be optimized but the potential large gain in interaction energy could be sufficient to allow for the replacement of the phosphonate-linker moiety.

Also within the scope of the present invention are complexes formed between a TS enzyme (as a whole or individual subunits) and the inhibitor of the invention. In one embodiment, the complex is not formed in its natural environment, ie., the organism or cell harboring the TS. Thus, the complex may be formed in vitro using isolated and purified TS or subunits thereof This complex is referred herein as “isolated.”

“Purification” of a TS or subunits thereof refers to the derivation of the protein or polypeptide by removing it from its original environment (for example, its natural environment). Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against the TS protein its subunits or against peptides derived therefrom can be used as purification reagents. Other purification methods are possible some of which are described in detail in the Examples. A purified polynucleotide or polypeptide may contain less than about 50%, preferably less than about 75%, and most preferably less than about 90%, of the cellular components with which it was originally associated. In one preferred embodiment, the TS or subunits thereof are substantially pure, which indicates the highest degree of purity which can be achieved using conventional purification techniques known in the art.

In another embodiment of the invention, the TS/inhibitor complex is formed in planta. In yet another embodiment, the complexes (formed in vivo or in vitro) do not contain inhibitors of formula I. For herbicide design purposes, the complex may be generated as a model, for example as a coordinate set for display on a computer graphics workstation for application of drug design algorithms, as described below.

Methods for Identifying Herbicidal Inhibitors

The invention further provides for methods for identifying novel TS inhibitors using (i) the high throughput compound screening, (ii) the structure-based approach and/or (iii) the homology approach.

A. High Throughput Screening

High throughput screening for identifying new inhibitors of TS may be used in an approach generally known in the art. The compounds to be tested in a high throughput assay may be synthesized and tested at random or the compounds may be selected based on the considerations outlined above. TS assays described in this specification may be used to test the activity of these compounds. An example of such an assay (complementation assay using E. coli mutants) is described in Example 6. However, any assay capable of detecting inhibition of the TS enzyme apparent to a person of skill in the art may be used.

B. Structure-Based Approach

The rational/structure-based design of novel inhibitors of TS, searching of chemical databases using known inhibitors or fragment thereof, methods of optimizing desired properties of the inhibitors (e.g., using the 3D structure of TS alone or in complex with the inhibitor) are also within the scope of the present invention.

To support the structure-based design and optimization of TS inhibitors, the following systems were established and are described herein: production of Salmonella and Arabidopsis TS subunits, TS assays including a novel microtiter plate TSβ-subunit assay, and protocols for crystallization of TS to improve X-ray diffraction patterns for improved resolution of 3D structures of the TS α-subunit (TSα). In addition, three-dimensional crystallized TS structures with inhibitors bound thereto were produced and methods for confirming the mechanism of action of designed inhibitors in planta were utilized.

TS Protein Production and Crystallization

TS may be produced, isolated and purified from any organisms that contains it, or contains a heterologous gene coding for it, using methods described herein or otherwise known in the art. As a matter of example, the mass production and purification of Salmonella TS is outlined below.

A 60 liter fermentation at 37° C. of E. coli strain CB149pSTB7 transformed with the plasmid pSTB7 carrying the Salmonella typhimiurium trpA and trpB genes was used to mass produce 320 g of cells which were overproducing tryptophan synthase. Washed cells were respended in 50 mM Tris-chloride, 5 mM EDTA, 0.1 mM pyridoxal phosphate, 10 mM mercaptoethanol (all adjusted to pH 7.8), and 1 mM phenylmethylsulfonylfluoride at 5 ml per grain of cells and homogenized by three passes through a Manton-Gaulin laboratory homogenizer (10,000 PSIG) for lysis of the cells. The lysate was centrifuged for 30 min at 17,500×G. A solution of 50 mM Tris-Cl, 5 mM EDTA, 0.1 mM pyridoxal phosphate, 10 mM mercaptoethanol (all adjusted to pH 7.8 with NaOH), 25 mM spermine and 30% PEG 8000 at a ratio of 2 parts to each 8 parts of lysate was added to the supernatant with mixing. The solution was immediately centrifuged at 17,500×G for 10 min, and the pellet discarded. The supernatant was incubated at 4° C. for 16 to 48 hrs until crystallization occurred. Crystals were collected by centrifugation at 17,500×G for 20 min, then were resuspended and washed with 50 mM Tris-chloride, 5 mM EDTA, 0.1 mM pyridoxal phosphate, 10 mM mercaptoethanol (all at pH 7.8), 6% PEG 8000 and 5 mM spermine with a second centrifugation at 17,500×G for 20 min. The crystals were resuspended in 50 mM bicine, 1 mM EDTA, 0.02 mM pyridoxal phosphate, and 10 mM mercaptoethanol (all adjusted to pH 7.8 with NaOH), and the solution warmed up to 37° C. to dissolve the crystals. The protein was then dialyzed overnight against 50 mM bicine, 1 mM EDTA, 0.02 mM pyridoxal phosphate, and 10 mM mercaptoethanol (all adjusted to pH 7.8 with NaOH) at 40° C., then centrifuged at 17,500×G for 25 min, and then at 27,500×G for 15 min. The supernatant was dialyzed for 23 hours against 0.1 M potassium phosphate buffer (pH 7.8), 5 mM EDTA, 0.2 mM pyridoxal phosphate, 10 mM mercaptoethanol, containing 85 g/L solid ammonium sulfate. The precipitate was recovered and resuspended in 10 volumes of the same ammonium sulfate buffer, and the suspensions were stored at −20° C.

Large crystals for crystallographic analysis may be prepared as described. A sample of the ammonium sulfate suspension was centrifuged and the precipitate was dissolved in 50 mM bicine buffer pH 7.8, 1 mM EDTA, 1 mM DTT, and 0.1 M pyridoxal phosphate, then dialyzed against the same buffer, loaded on a MonoQ column and eluted with a gradient of 0 to 1 M NaCl. The two protein peaks that eluted were combined and a small amount mixed with an equal volume of well solution (50 mM bicine buffer pH 7.8, 1 mM EDTA, 1 mM DTT, 12% PEG 8000, 0.08% sodium azide, and 21% spermine) and placed on a post in the well, to allow large crystals to grow. Large crystals may be later cut to a smaller size for enzyme structure determination.

Plant TS enzyme and/or its subunits may be partially purified from plant tissues (as described in Example 4) or from recombinantly expressed plant TS subunits in E. coli or other organism suitable for overexpression of the plant protein (as described in Example 5). Any modification of these methods obvious to a person of skill in the art and/or equivalent thereto is considered to be within the scope of the present invention.

In one embodiment of the invention, the plant TS is partially purified at least about 10 fold, and most preferably at least about 180 fold. This partial purification method comprises (i) homogenizing plant tissue; (ii) centrifuging the plant homogenate; (iii) mixing the supernatant obtained in step (ii) with ammonium sulfate from about 25 to about 35% of saturation and subjecting it to centrifugation; (iv) collecting the supernatant obtained after centrifugation in step (iii) and mixing it with ammonium sulfate from about 45% to about 60% of saturation and subjecting it to centrifugation; and (v) collecting the precipitate containing purified TS. In another embodiment, a single precipitation step by ammonium sulphate about 80% to about 90% of saturation may be used. In one embodiment, the method further comprises applying the dissolved precipitate from step (v) to Waters SW300 column or equivalent thereof.

Protein-Based Lead Finding and Optimization

In one aspect of the invention, methods for identifying novel herbicide inhibitors using the known structure of the TS enzyme are provided. The methods rely on the X-ray structures or protein models of the entire TS molecule, or alternatively on the models of the active sites alone. These methods are described in more detail below.

Molecular Graphics, Electrostatics Calculations, and Surfaces

Disclosed is a method of displaying the coordinates, molecular surfaces and mapping of physicochemical properties onto the atoms or surfaces to generate a meaningful description of the inhibitor binding site of the protein. Into this binding site, small molecules may be placed, by for example replacement of existing molecules at that site, using an alignment of the new molecule to be placed into the site onto the molecule co-crystallized with the TS protein or previously modeled or docked into the TS protein binding site.

For purposes of the present invention, the molecule co-crystallized with TS or modeled or docked into the TS binding site is the “template inhibitor”. The “target inhibitor” is a new molecule to be placed into the TS binding site in place of the template inhibitor. All programs cited herein are described by their respective documentation. If not specified, parameters are chosen to be the values provided by the program setup, as provided by the vendor or within reasonable ranges. Acceptable ranges to the parameter settings are known to those skilled in the art.

The alignment of the template inhibitor and the target inhibitor may be generated by computer programs such as Alignment, CatShape, APEX (Molecular Simulations Inc. (MSI), 9685 Scranton Rd., San Diego, Calif.) or similar, or by overlaying analogous features of the inhibitors, such as (partially) charged groups, hydrogen bond donors/acceptors, hydrophobic portions, such as an alkyl chain or an aromatic group.

Alternatively, or in addition, molecules can also be placed into the enzyme active site using an interactive modeling graphics program or methods know in the art, such as docking, using the computer programs Affinity, LUDI, or Receptor (MSI). A program such as CatShape can not only be used to align molecules but, as described in the manual (Catalyst 4.0, MSI), search for novel molecules that fit into the binding site. A template from the shape search can be generated, in addition to the method described above, by using a program such as LUDI to position various fragments into the TS binding site. An overlay of all fragments that fit into the binding site may then be used to generate a receptor surface using the program Receptor (MSI). This receptor model is useful for aligning molecules reported in an electronic database into the binding site. Examples of such databases are proprietary compound databases, e.g. Cyanamid's CL-File, the Available Chemicals Directory (ACD) (distributed by MSI), and virtual chemical libraries using appropriate programs such as Catalyst.

Once an initial positioning of the molecule of interest in the binding site has been found, potential energy function based methods well known in the art, such as energy minimization, molecular mechanics, molecular dynamics or Metropolis Monte Carlo (MMC) methods may be used to refine the position of the small molecule in the binding site, preferably by allowing flexible rearrangements of the protein or parts thereof.

The resulting energetically best conformations and orientations may be compared to the binding of other previously identified inhibitors. Interaction energy values from the force filed calculations, overall fit of the binding site and additional criteria such as satisfying hydrogen bonds and dipolar and charge interactions, as for example, implemented in the programs LUDI and DOCK, may be used to gauge the quality of the inhibitor. Inhibitors with a better score or lower interaction energy are candidates that are expected to have improved binding properties.

Introduction of other modifications, such as elements to rigidify the conformations, thereby reducing the difference in entropy of free and bound state, or, for the same reason, removal of hydrophilic groups can also be studied using the above described docking/refinement methods.

In order to improve on a new inhibitor, additional groups can be added to the inhibitor. This can be done manually using an interactive molecular graphics program followed by the above described potential energy function-based refinement methods or using a rule or score-based system, for example as implemented in the program LUDI (MSI).

In one approach, a core molecule is chosen and various test fragments from a database library are modeled into the core molecule with an objective to improve the number and strength of intermolecular interactions.

This method comprises the steps of (i) using a crystal structure of TS (or a comparable model of a TS protein or TS active site) to define a center of the search at a position where a small molecule should bind to inhibit TS activity (for example, the active site of either subunit, the “tunnel”, or at a location close to the portion of the protein that is known to rearrange upon binding of substrates); (ii) performing an analysis of this binding site in terms of interaction sites (for example, electron and hydrogen bonding acceptors and donors, hydrophobic surfaces, electrostatic potentials); (iii) searching for small molecules in chemical databases that completely or partially complement the previously defined interaction sites; (iv) fitting those “hits” into the binding site and evaluating the score or energy value for the binding strength; and (v) selecting candidates for synthesis and testing: according to various criteria, such as availability, ease of synthesis, or calculated physicochemical parameters (e.g. clogP) of the compound.

Inhibitor-Based Lead Optimization

In another embodiment of the invention, methods for identifying inhibitors based on the structural information about the known inhibitors are provided. This approach is known as a rational design based on TS-bound molecules.

This method includes (i) analyzing the conformation of the inhibitor in the crystal structure of the TS-inhibitor complex and (ii) designing compounds that mimic inhibitors and designing improved properties of designed compounds (“mimics”). Specifically, the method comprises searching an electronic database with a known inhibitor or a portion thereof, or its computer representation (i.e., an abstraction of the molecule as a pharmacophore model) as a search template. Alternatively, or in addition to the database searches, modifications of the inhibitor may be designed so that the overall positions of groups essential for binding to TS are preserved, but other atoms of groups are modified, omitted, or added. Groups that are important for binding to TSα have been described above and in Example 18.

C. Homology Modeling

In this method, the crystal structure of the Salmonella TS enzyme may be used as a template to generate a homology model of TS from another source, such as a higher plant (provided that the amino acid sequence of the plant protein is known). Any other known TS enzyme may be used as a template. The advantage of homology models is that inhibitor/protein designs can be designed directly on the protein/gene that is being targeted for inhibition or modification. For example, this approach can be used to show that binding sites in Arabidopsis TS are equivalent to those in Salmonella TS.

The process of homology modeling of a protein having TS activity by protein homology modeling techniques may be performed using one or more known (from crystallographic analysis or homology modeling) 3D structures of TS or structural homologues thereof. Using the same process, TS fragments involved in forming the inhibitor binding site could be modeled (instead of a complete TS molecule). The process of modeling typically includes (i) selection of one or more template molecules, (ii) alignment of the amino acid sequence of the template protein(s) with the amino acid sequence of the target protein, (iii) generating a computer model of the target protein using protein homology. Optionally, the computer model generated in step (iii) may be additionally refined using potential energy or scoring functions with minimization, molecular dynamics, or Monte-Carlo methods.

Computer models are useful for understanding the mode-of-action and inhibition of TS. The inhibitors may be designed based on these homology models. This knowledge can then be used, in conjunction with interactive molecular graphics methods, database searching methods, de-novo design methods, or similar approaches known in the art, to improve desired properties of the inhibitors (for example, binding activity or preferred sites for chemical modifications, that can introduce desired physicochemical or other properties that increase herbicidal efficacy).

A structural homologue is a protein or protein model that has essentially the same fold, wherein fold is the relative orientation of secondary structural elements such as β-sheets and/or α helices relative to each other in three-dimensional space. For the Tsα subunit, the fold is characterized as a β-barrel structure.

TS Assay Methods

To test the specificity and efficacy of inhibitors designed or identified according to the methods of the invention, in vitro enzyme assays may be used. These assays are also useful for characterizing variant forms of the TS enzyme, such as herbicide resistant mutant TS enzymes, as well as characterizing TS enzymes isolated from various sources, for example, from E. coli cultures expressing TS, from crops and weed species. Any testing method known in the art may be used. For example, assays described in Smith O H and Yanofsky C 1962 Methods in Enzymology vol. V pp 794-806, or more preferrably pp 801-806 (Tryptophan synthetase); Creighton T E and Yanofsky C Methods in Enzymology vol. XVIIA pp 365; Kirschner et al. 1975 Eur. J. Biochemistry 60:513; J. Biol. Chem. 240:725 (1965) Hardman and Yanofsky; J. Biol. Chem. 241:980 (1966); J. Biol. Chem. 245:6016-6025 (1970); J. Biol. Chem. 246:1449 (1971); J. Biol. Chem. 253:6266 (1978); J. Biol. Chem. 262:10678.

The assays described in the Examples may also be used.

Inhibition of either the α or the β reaction of tryptophan synthase inhibits the activity of the holoenzyme. To measure the inhibition of TS, one can either measure the reduction in activity of the TSα reaction or of the TSβ reaction. However, quantification of the activity of TSα requires a pure enzyme. This is because the necessary substrate, IGP, has a phosphate group that is particularly labile in the presence of non-specific phosphoesterases. As a result, impure enzyme preparations that contain competing enzyme activities generally obscure the true activity of TSα by reducing the apparent concentration of the substrate.

Due to a phenomenon known as cooperativity, each subunit reaction, TSα or TSβ, is known to be most active when the subunits are combined in the holoenzyme, α₂β₂. The TSα activity is quantified for the intact holoenzyme by adding limiting IGP in the presence of excess serine, serine being required for the TSβ reaction. Glyceraldehyde3-P (G3P) is measured as the product instead of tryptophan but for G3P to be produced, an equal amount of tryptophan also had to have been produced. G3P is measured in a reaction coupled to NADH production via commercial glyceraldehyde 3-phosphate dehydrogenase, another highly purified enzyme.

Since plants have relatively low levels of endogenous TS, it has proven difficult to purifiy plant TS to homogeneity. This means that the TSα activity from plants cannot be reliably assayed, because the assay requires a highly purified enzyme and crude plant enzyme preparations may contain a number of interfering enzymes. Instead, endogenous TS activity in plants is measured by the TSβ reaction. This allows a determination of the parts of the plants where TS activity is the most concentrated and the developmental growth stage of plants when TS is the most active. The TSβ reaction does not require pure enzyme, but for accuracy does require a careful separation of the substrate indole and the product tryptophan, the absorption spectra of which are highly overlapping. In a preferred assay for TSβ activity, the disappearance of indole is measured in the presence of excess serine, which occurs in the production of tryptophan. The assay is quantified by the time dependent reduction in indole. The assay is described in more detail in Example 4.

A novel method for testing the TSβ reaction is provided. The method comprises isolating and quantifying indole via a microtiter plate assay utilizing a three-phase liquid system. In this method, a crude homogenate from plant tissues or a partially purified ammonium sulfate fraction from the crude plant homogenate is used as a source of the plant enzyme. The method comprises (i) conducting the TSβ reaction in the presence of the plant TS, indole and serine; (ii) separating the indole containing phase and transferring it into the microtiter plate to form a three-phase liquid system as described in Example 4; and (iii) determining the amount of indole.

An improved assay for TSα reaction is also within the scope of the present invention. The assay is adapted to the microtiter plate format, which conserves reagents and allows simultaneous observation of kinetic enzyme assays. In addition, the level of the IGP substrate in the reaction is less than 5× the Km of the enzyme for IGP and preferably from about 1× to about 2×. In one embodiment, when weak inhibitors are tested in this TSα assay, the inhibitor is pre-incubated with the enzyme substantially before the competing substrate is added.

Reversal Assay

Inhibition of plant TS in vivo may be verified by demonstrating reversal of herbicidal symptoms by supplementing treated plants with tryptophan. The term reversal is conceptually and in practical terms equivalent to the rescue from, complementation to, and prevention of injury. Only those inhibitors whose effects can be overcome with tryptophan are within the scope of the invention. The reversal assay represents a mechanism-based assay for identification of herbicidal inhibitors. An example of such an assay is provided in the Examples. However, any modifications know or obvious to those of skill in the art may be used.

Methods for Identifying and Constructing Herbicide Resistant TS

Also within the scope of the present invention are methods for designing herbicide resistant TS in plants of commercial importance, such as for example corn, soybean, canola, sugar beet, sugarcane, barley, wheat, rice, and other crop plants. The TS variant proteins constructed according to these methods and transgenic plants expressing the variant TS protein are within the scope of the present invention.

The molecular interactions between herbicidal inhibitors of the invention and the target protein, TS, can be used to design alterations in the protein to inhibit binding. Structure based design has been shown to be an effective approach to design herbicide tolerant genes (Ott et al. 1996, JMB 263:359 and U.S. Pat. No. 5,853,973 to Kakefuda et al.). The same approach, or any other approach obvious to a person of skill in the art, may be used to design and make TS variant proteins resistant to the herbicidal inhibitors of the invention. Briefly, homology models, or, for the most part, sequences of genes or proteins of TS can be used to derive potential herbicide resistance sites. This requires the mapping of sites involved in binding the inhibitor, or sites that are involved in the transport of the inhibitor to the binding site, or sites that are involved in the subunit communication onto the sequence, or, by visual or computational analysis of the 3D structures (Cartesian or internal coordinates of the protein structures). The sites that have been identified to be involved in the mechanism of binding the inhibitor can then be experimentally mutated using molecular biology techniques known in the art. In one embodiment of the invention, at least one of the following amino acids are mutated: αL100, αY102, αA129, αI153, αL177, αF212, in the α-subunit, and βI326 and βP318 in the β-subunit of Salmonella. Various mutations at those positions into other amino acids are generated and expression of these mutant proteins in heterologous expression systems and determination of their activity with and without inhibitor can be used to further select TS protein variants with a desired profile, e.g. resistance against inhibition by a chosen herbicide. Alternatively, resistance genes can be tested in vivo by transformation in plants. Further refinement of the mutation, inlcuding combining various mutations can be used to iteratively improve the desired enzyme characteristics.

In one embodiment, screening for herbicide resistant variants can be done using an E. coli mutant strain that lacks expression of its endogenous TSβ (or TSα) subunit. It is known that this mutation can be complemented with a plasmid expressing the Arabidopsis TSβ (or TSα)-subunit as described in Example 6. This E. coli strain may be used in the method of the present invention to screen for plant, for example, Arabidopsis TSβ mutants that are resistant to compounds that inhibit TS activity. This process can similarly be performed for screening for variants of TSα that are resistant to TS inhibitors. (E. R Radwanski, J. Zhao, R. L. Last, Mol Gen Genet [1995] 248:657-667).

The resistant TS variant proteins and their encoding genes identified using the methods described above are also within the scope of the invention. The genes conferring resistance to TS inhibiting herbicides may also be used to produce transgenic crop plants using methods well known in the art.

Methods of Weed Control

The invention further provides for methods of weed control by applying the herbicidal inhibitors of the invention. The mode of application and the amount of the inhibitor utilized is as known in the art. For example, the inhibitors may be used for postemergence control of a variety of undesirable plant species and may be applied to the foliage or stems at rates from about 0.5 kg/ha to about 10 kg/ha as described in U.S. Pat. No. 5,635,449.

The invention is further described in the following non-limiting examples.

### EXAMPLES

### Example 1

Initial attempts to identify inhibitors of TS are described in this example. Phosphonate isosteres of a known inhibitor indole-3-propanol phosphate (IPP) were synthesized and tested for TS inhibitory activity and herbicidal potency.

IPP is an inhibitor of TSα subunit reaction with a Kᵢ of 15 μM. In the following experiments, the activity of IPP was compared with two potential inhibitors (phosphonates7a and 7b) prepared according to Scheme 1.

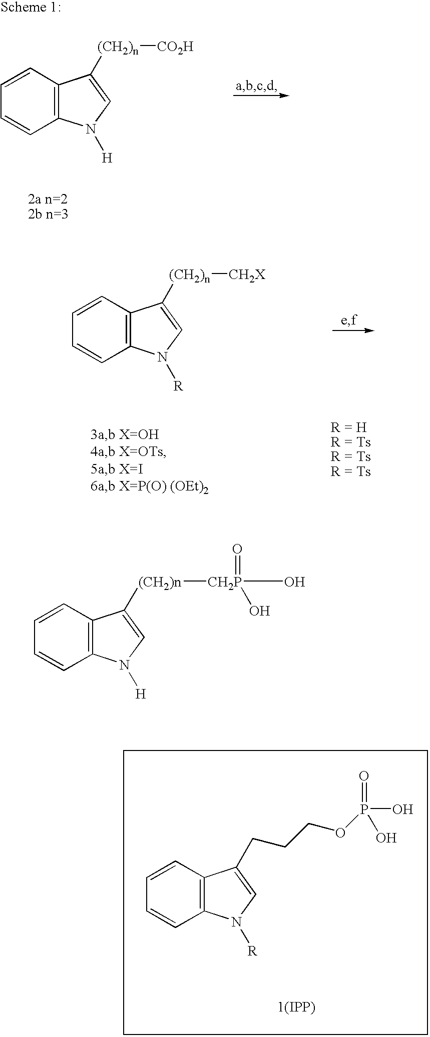

Reagents and conditions: (a) LAH; (b) NaH, TsCl; (c) NaI; (d) P(OEt)₃; (e) 20% KOH; (f) TMSBr

Referring to Scheme 1, reduction of 3-indole-propionic acid, 2a, and 3-indole-butyric acid, 2b, with LAH provided the primary alcohols 3a and 3b. These were converted to ditosylated derivatives 4a and 4b by treatment with 2 equivalents each of sodium hydride and tosyl chloride. Conversion to the primary iodide followed by treatment with triethylphoshite yielded the desired phosphonate esters 6a and 6b. Removal of the protecting groups gave the desired phosphonates 7a and 7b.

The targeted compounds were tested both in vitro for inhibition of the TSα subunit reaction and in vivo for herbicidal activity on whole plants. The tests were conducted as described in Example 3 (in vitro assay) and Example 2 (herbicidal activity). These results are shown in Table 1.

TABLE 1

| Compound | I₅₀ TS (μM)* | Herbicidal activity** |
|---|---|---|
| 1 | 5 | inactive |
| 7a | 125 | inactive |
| 7b | 20 | weak |

*Determined via the TSα reaction of the highly purified Salmonella typhimiurium holoenzyme.The I₅₀ is the concentration required for 50% inhibition of the enzyme activity in the absence of the inhibitor.
**inactive = no activity at 4 kg/ha in a post emergence greenhouse test; weak activity = maximum 20-30% injury on any species

The I₅₀ value represented in Table 1 is a measure of enzyme activity and indicates the concentration of inhibitor which is able to reduce the in vitro enzyme activity by 50% under the conditions of the assay described below. This is a common means by which inhibitor effects on enzymes are compared.

As shown in Table 1, phosphonate 7b was found to be an inhibitor of TS with an slightly weaker I₅₀ than I₅₀ for the corresponding phosphate IPP. The shorter chain phosphonate analog 7a was a weaker inhibitor than 7b. In greenhouse testing, only compound 7b showed any activity. This compound slightly inhibited the growth of one plant species when applied postemergence. This effect was minimal and the plants were able to grow out of the early symptoms.

### Example 2

With an intention to produce stronger TS inhibitors, a new set of test compounds was prepared.

In this experiment, compounds having a shape similar to the reactive intermediate (compound 8 shown below) of the TSα subunit reaction were prepared. In the TSα enzymatic reaction, the C-3 position of the indole ring of the IGP substrate is protonated resulting in a reactive intermediate 8 containing an sp₃ atom at position C-3. The hypothesis tested in this experiment was the C-3 at this position may be important for the interaction with the enzyme. Thus, test compounds were constructed with an sp₃ atom that mimics the C-3 position of the reaction intermediate 8. In addition, the C-2 atom of the indole ring found in the IGP substrate, as well as in the known inhibitor IPP, was removed. This was done to simplify the synthesis and to obtain compounds having a higher conformational flexibility than the original substrate. The test compounds are represented by the generic formula 9.

The designation sp3 is well known in the art and refers to an atomic and molecular orbital formed by combination of p- and s-orbital, which are charged clouds around atoms that extend out in space in direction of other atoms and point to the corners of a regular tetrahydron. “Advanced Organic Chemistry”, Jerry March, ed., John Wiley and Sons, Interscience Publication.

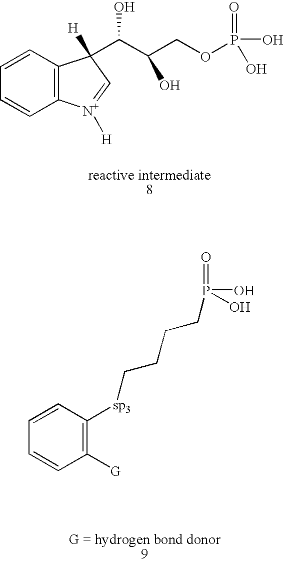

The first set of compounds of formula 9 that were prepared and tested were arylalkylphosphonate sulfides (sp₃=S) bearing either a carboxamide or amine in the ortho position to the sulfur atom. The synthesis of these compounds is described in Scheme 2. The key reactions were an arylmercaptide addition to diethyl 4-bromobutylphosphonate followed by TMSBr cleavage of the esters.

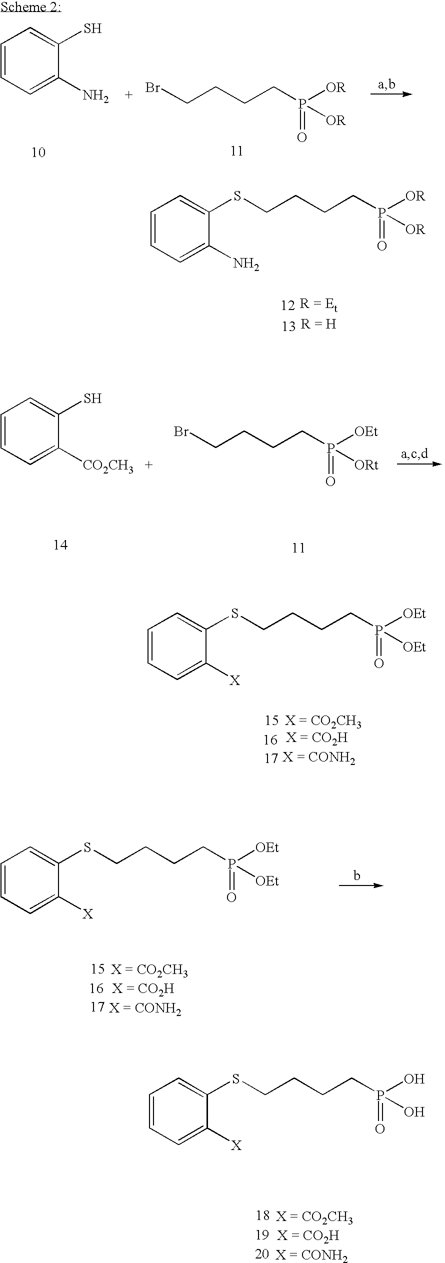

Reagents and conditions: (a) TEA; (b) TMSBr; (c) NaOH; (d) SOCl₂, NH₃

The four phosphonic acids (13, 18, 19, 20) shown in Scheme 2 were tested in the in vitro TS enzyme assay. Although compounds 18-20 were inactive, the ortho-amino compound 13 had very good enzymatic activity (I₅₀=400 nM) in the in vitro assay as shown in Table 2. In addition, this compound and its related salts and esters displayed greenhouse herbicidal activity as shown in Table 2.

The herbicidal activity of the compounds was tested as described in the U.S. Pat. No. 5,635,449. Specifically, the herbicidal activity of the compounds of the present invention is demonstrated by the following tests, wherein a variety of dicotyledonous and monocotyledonous plants are treated with test compounds, dispersed in aqueous acetone mixtures. In the tests, seedling plants are grown in jiffy flats for about two weeks. The test compounds are dispersed in 50/50 acetone/water mixtures containing 0.5% TWEEN®20, a polyoxyethylene sorbitan monolaurate surfactant of Atlas Chemical Industries, in sufficient quantities to provide the equivalent of about 1.0 kg to 8.0 kg per hectare of test compound when applied to the plants through a spray nozzle operating at 40 psi for a predetermined time. After spraying, the plants are placed on greenhouse benches and are cared for in the usual manner, commensurate with conventional greenhouse practices. From four to five weeks after treatment, the seedling plants are examined and rate according to the rating system set forth below.

| | | % Control |
|---|---|---|
| Rating | Meaning | Compared to Check |
| 9 | Complete Kill | 100 |
| 8 | Approaching Complete Kill | 91-99 |
| 7 | Good Herbicidal Effect | 80-90 |
| 6 | Herbicidal Effect | 65-79 |
| 5 | Definite Injury | 45-64 |
| 4 | Injury | 30-44 |
| 3 | Moderate Effect | 16-29 |
| 2 | Slight Effect | 6-15 |
| 1 | Trace Effect | 1-5 |
| 0 | No Effect | 0 |
| — | No Evaluation | |

The discovery of the good enzymatic and herbicidal activity of the aryl sulfide 13, prompted the synthesis of additional analogs. Scheme 3 shows the synthesis of several ortho-hydroxy phenyl sulfides. The compound 28 was made by treatment of aldehyde 25 with the anion of tetraethyl methylendiphosphonate (Kosolapoff, G. J. Amer. Chem. Soc. 1953, 75, 1500). This Wittig reaction afforded the trans olefin selectively. The sulfoxide and sulfone derivatives were prepared by oxidation of phosphonic acid. Purification of these very polar compounds required the use of C-18 reverse phase chromatography.

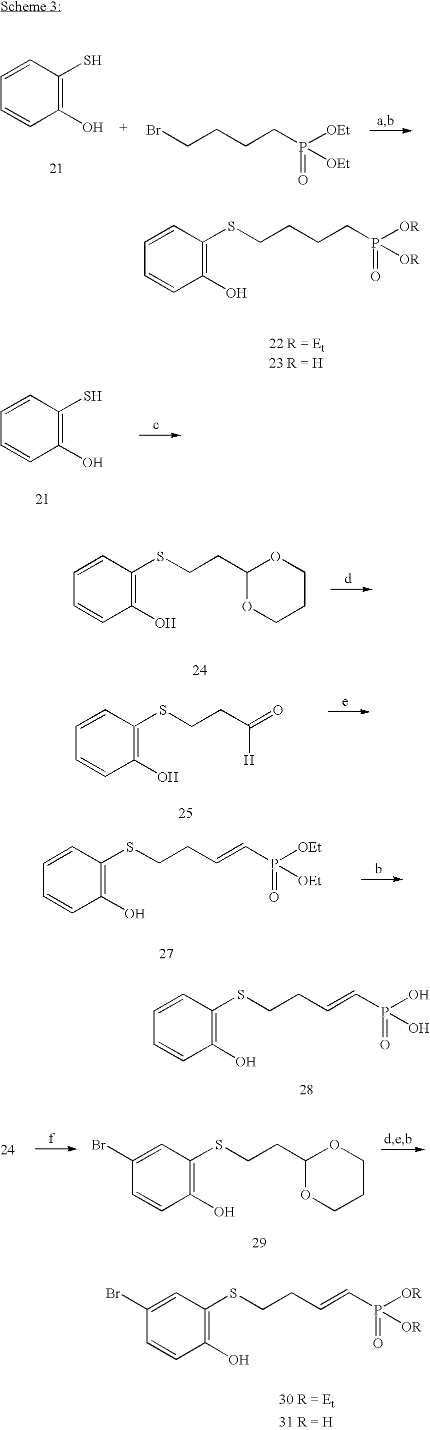

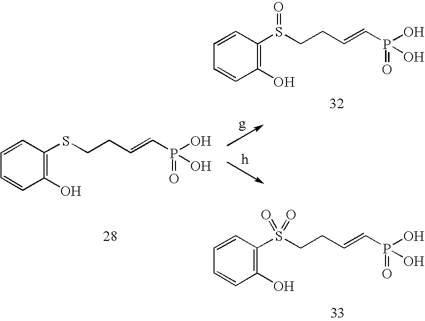

Reagents and conditions: (a) TEA; (b) TMSBr; (c) TEA, 2-(2-chloroethyl)-1,3-dioxane; (d) HCL; (e) nBuLi, CH₂(P(═O)(OEt)₂)₂; (f) Br₂; (g) 1 equiv. mCPA; (h) 2 equiv. mCPBA

Table 2 compiles the biological activity data for the tested aryl sulfide phosphonates. The herbicidal activities of several ortho-hydroxyphenyl sulfides was improved compared to compound 13. For all compounds, only postemergence herbicidal activity was observed. Also, introduction of rigidity in the linking chain in the form of a double bond improved biological activity (compound 28).

TABLE 2
Aryl sulfide phosphonate inhibitors of TSα

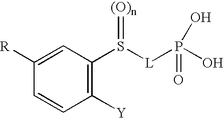

| | | | | | I₅₀ TS | Herbicidal |
|---|---|---|---|---|---|---|
| Cmpd # | n | L | R | Y | (nM) | Activity** |
| 13 | 0 | —(CH₂)₄— | H | NH₂ | 400 | + |
| 22 | 0 | —(CH₂)₄— | H | OH | 130 | +++ |
| 28 | 0 | — | H | OH | 570 | ++++ |
| | | CH₂CH₂CH═CH | | | | |
| | | — | | | | |
| 31 | 0 | — | Br | OH | 260 | + |
| | | CH₂CH₂CH═CH | | | | |
| | | — | | | | |
| 32 | 1 | —(CH₂)₄— | H | OH | 440 | +++ |
| 33 | 2 | —(CH₂)₄— | H | OH | 360 | IA |

See footnote to Table 1 for legend.; IA = inactive
**Postemergence application. Herbicide rating scale + = 30-80% injury to one species;
++ = 80% to 100% injury to one species;
+++ = 80-100% injury to two species;
++++ = 80-100% injury to more than three species.

Plants treated with tryptophan synthase herbicides showed symptoms typical of a herbicide whose mode of action is the inhibition of amino acid biosynthesis. The herbicidal activity was slow to develop, beginning with growth cessation, chlorosis or mottling, followed by some necrosis. Herbicidal profiles for selected compounds are represented in Table 2.

### Example 3

This examples shows inhibition of the Salmonella TSα by some of the inhibitors of the invention. The enzyme activity was measured using a pure enzyme. The term “pure” indicates the highest degree of purity that can be achieved by purification methods known in the art. Alternatively, TS is “pure” if two single protein bands can be observed by SDS polyacrylamide gel electrophosresis and Coomasie Brilliant Blue R250 staining at increasing concentrations of total protein. The methods were used, and the materials were prepared, as described below.

Small Scale Production and Purification of Salmonella TS for Inhibitor Assays

A system for small scale production of Salmonella TS was developed to employ enough enzyme for in vitro assays. E. coli strain CB149pSTB7 (described in Kawasaki et al., J. Biol. Chem. 262:10678, 1987) was a gift of Edith Miles, National Institutes of Health was used to overproduce Salmonella tryptophan synthase (TS). The multicopy plasmid pSTB7 containing Salmonella typhimiurium genes for trpA and trpB (as described in the above Kawasaki et al. publication), encoding the α and β subunits of tryptophan synthase, respectively, was used.

E. coli cells grown with shaking at 37° C. in L-broth (1% tryptone, 0.5% yeast extract, 1% sodium chloride, 0.1% glucose adjusted to pH 7) supplemented with 30 mg/L ampicillin were transferred to induction medium at either 28° C. or 37° C. for 24 hrs. The induction medium contained Minimal Medium (0.8 mM magnesium sulfate×heptahydrate, 10 mM citric acid×monohydrate, 60 mM dibasic potassium phosphate 10 mM monobasic sodium phosphate, 10 mM monoammonium phosphate, (all adjusted with NaOH to pH 6.6), 0.5% glucose, 0.5% casein hydrolysate, 5 mg/L tryptophan, plus 30 mg/L ampicillin. At the end of the growth period, cells were collected by centrifugation (10,000×g), resuspended in 15 mL (2.5% of the original medium volume) of 0.85% sodium chloride, and centrifuged again.

To extract the TS, cells were resuspended in 4 mL of 50 mM Tris-chloride, 5 mM EDTA, 0.1 mM pyridoxal phosphate, 10 mM mercaptoethanol (all adjusted to pH 7.8 with HCl), and 1 mM phenylmethylsulfonylfluoride, to which was added 0.6 mg/mL lysozyme, and the cells were sonicated (3 bursts of 15 sec). The debris was removed by centrifugation at 27,000×g for 20 min, and the supernatant was transferred to a new tube. To this was added, with gentle mixing, 1 mL of 50 mM Tris-Cl, 5 mM EDTA, 0.1 mM pyridoxal phosphate, 10 mM mercaptoethanol (all adjusted to pH 7.8 with NaOH), 25 mM spermine and 30% PEG 8000. Following immediate centrifugation for 5 min at 27,000×g, the supernatant was collected and incubated for 16 to 48 hrs at 4° C. until crystals were formed.

Crystals were collected by centrifugation at 4-5° C. for 15 min at 27,000×g, and then were washed with 50 mM Tris-chloride, 5 mM EDTA, 0.1 mM pyridoxal phosphate, 10 mM mercaptoethanol (all at pH 7.8), 6% PEG 8000 and 5 mM spermine with recentrifugation. Crystals were resuspended and stirred at 37° C. for 10 min in 1 mL of 50 mM bicine, 1 mM EDTA,0.02 mM pyridoxal phosphate, and 10 mM mercaptoethanol (all adjusted to pH 7.8 with NaOH), then were dialyzed overnight at 4° C. against 100 mL of the same pH 7.8, 50 mM bicine, 1 mM EDTA, 0.02 mM pyridoxal phosphate, and 10 mM mercaptoethanol solution. The protein dialysate was centrifuged in a microfuge 6 min at 12,000×g and the pellet was discarded. The supernatant was subsequently dialyzed against 0.1 M potassium phosphate buffer (pH 7.8), 5 mM EDTA, 0.2 mM pyridoxal phosphate, 10 mM mercaptoethanol, supplemented with 85 g/L solid ammonium sulfate to precipitate TS. The precipitate was collected by centrifugation, washed once with the ammonium sulfate-phosphate buffer, centrifuged again, resuspended in ammonium sulfate-phosphate buffer and stored at −20° C. Purity of TS was established by SDS gel electrophoresis using increasing protein loads. The results on the gel showed only two protein components, representing the subunits TSα and TSβ.

Synthesis of Indole Glycerol Phosphate

IGP, the substrate for the forward TSα reaction, was not commercially available, but was biosynthesized by the reverse reaction of TSα (indole+D-glyceraldehyde 3-P →indole-3-glycerol-phosphate). The reaction was monitored by the disappearance of indole from the reaction mix.

Any method suitable for synthesizing IGP and separating IGP from substances that would interfere in the assay could be used. (For example, Smith O H and Yanofsky C Methods in Enzymology vol. VI pp 590-597; or Brzovic P S, Ngo K N, Dunn M F 1992 Biochemistry 31:3831-3839).

DL-glyceraldehyde-3-phosphate was prepared according to the distributor's method (Sigma Chemical Co., St. Louis, Mo.) from the barium salt of the diethylacetal with the final solution adjusted to pH 4 with NH₄OH. IGP was prepared in a solution containing TS (approximately 0.2 to 0.3 mg/ml), 5 mM EDTA, 50 mM potassium phosphate buffer at pH 7.3, 6 mM indole, and approximately 10-13 mM glyceraldehyde-3-phosphate with incubation at 25° C. to 37° C. for up to 16 hrs. Utilization of indole was unaffected by pH in the range of 5.3 to 7.3 after 1 hr of incubation at 25° C. or 37° C., while utilization after 16 hrs was about 97% at pH 5.3., about 94% at pH 6.3, and about 85 to 88% at pH 7.3. Disappearance of indole was monitored at a wavelength of 540 nm (A₅₄₀)or of 567 nm (A₅₆₇)after a 30 to 60 min reaction, using 12.8 g/l dimethylaminobenzaldehyde, 64 ml/l concentrated HCL, in ethanol, and up to 14% aqueous sample by volume. IGP was separated from indole by conventional ion-exchange chromatography, by HPLC (Waters C18-Zorbax column, Waters Corporation, Franklin Mass., 0 to 80% acetonitrile, 1 ml/min), or preferably using a C18 Sep-Pak cartridge (Water Corporation, Franklin, Mass.) (IGP is in the aqueous flow-through) and evaluated by HPLC. IGP was separated from G3P by the method of Brznovic et al., 1992, cited above. G3P was monitored using G3P dehydrogenase, and IGP by the periodate method wherein the 100 μl test solution, or IGP, was mixed with 60 μl 0.66 M acetate buffer pH 5 containing 33 mM sodium metaperiodate for 20 min then treated with base (80 μl 1N NaOH) and partitioned into 1 ml ethylacetate and the absorbance monitored at 290 nm.

Assays for Testing Inhibition of Tryptophan Synthase by the TSα-Reaction

Inhibitors of TS were identified by their ability to inhibit the production of glyceraldehyde-3-P by the TSα reaction of the Salmonella typhimurium holoenzyme (α₂β₂) in the presence of a limiting amount of indole-3-glycerolphosphate and an excess of serine

The assay was developed as a new microtiter plate kinetic enzyme assay based on the combined methods of Creighton (EurJBch 13:1-10, 1970) and Creighton and Yanofsky (JBC 241:980, 1966) with modifications. The rate of glyceraldehyde production was measured as the linear depletion of NAD+ (spectrophotometric absorbance at 340 nm) in the presence of glyceraldehyde-3-phosphate dehydrogenase in a coupled enzyme assay.

The assay solution contained a test inhibitor compound, 50 mM Tris-Cl (pH 7.8), 6 mM sodium arsenate, 5 μg/ml pyridoxal phosphate, 0.5 mM DTT, 0.18M NaCl, 60 mM serine, 1.6 mM NAD+, 8 e.u./ml yeast glyceraldehyde-3-phosphate dehydrogenase (Sigma, Catalog #G2647; Kirschner et al., Eur J Bch, 1975, 60:513 and approximately 1.5 e.u. Salmonella TS. 100 μM IGP was added to start the reaction, which was run at 37° C. and using 300 μl per assay in a microtiter plate.

The substrate IGP was used at 1.5 to 2 times its Km concentration to enhance the likelihood of identifying weak inhibitors, binding at the substrate binding site. This approach to identifying enzyme inhibitors was novel, since an excess of all substrates, (at least 5-times the Km value of each), is conventionally used in the measurement of enzyme activity.

Potential inhibitors were evaluated by adding 100 μM inhibitor (equimolar to substrate IGP):or less, in a 1:1 dilution series down from 100 μM, until the inhibition measured was less than 15%. Reaction rates at Vmax were compared in the presence and absence of inhibitors.

In addition, some weaker inhibitors were identified following preincubation of the inhibitor with the TS assay mix for 24 hrs prior to the addition of IGP. The identifying weaker inhibitors is to aid in a quantitative structure activity relationship (QSAR) evaluation, or to identify new herbicidal inhibitor leads. The previously known inhibitor IPP was used as a standard in all tests, where the I₅₀ for IPP was 1-2 uM.

The results of the in vitro assay are represented in Table 3. The first two inhibitor compounds show typical data from which the I₅₀ values were calculated.

TABLE 3

| | | | Enzyme |
|---|---|---|---|
| | | | activity, |
| | TS inhibition | Concentration | % of |
| Structure | I₅₀ nM* | nM | control |
| No inhibitor | — | — | 100 |
| Phosphonic acid, | 70 | 1000 | 8.7 |
| {4-[2-amino-5- | | | |
| bromophenyl)thio]butyl}- | | | |
| | | 300 | 24.7 |
| | | 100 | 38.5 |
| | | 30 | 70.5 |
| Phosphonic acid, | 250 | 10000 | 4.5 |
| {4-[(o-aminophenyl)thio]-2- | | | |
| butenyl}- | | | |
| | | 3000 | 13.8 |
| | | 1000 | 25.3 |
| | | 300 | 43.4 |
| | | 100 | 70.7 |
| Phosphonic acid, | 400 | | |
| {4-[(o- | | | |
| aminophenyl)thio]butyl}-, | | | |
| compound with | | | |
| cyclohexylamine (1:2) | | | |
| Phosphonic acid, | 400 | | |
| {4-[(o- | | | |
| aminophenyl)thio]butyl}-, | | | |
| dilithium salt | | | |
| indolepropanol phosphate | 2000 | | |
| (IPP) | | | |
| Phosphonic acid, | 5000 | | |
| {4-[3-amino-2- | | | |
| phridyl)thio]butyl}- | | | |
| Phosphonic acid, | 7000 | | |
| {4-[(2-amino- | | | |
| alpha,alpha,alpha-trifluoro-p- | | | |
| tolyl)thio]butyl}- | | | |
| Phosphonic acid, | 20000 | | |
| [4-(indol-3-yl)butyl]-** | | | |
| Phosphinic acid, | 100000 | | |
| {4-[(o- | | | |
| aminophenyl)thio]butyl}methyl- | | | |
| *TS activity was measured with the TSα raction using the Salmonella holoenzyme. The assay was quantified at a steady Vmax rate A₃₄₀ in a 30 min assay at 37° C. | | | |
| # The reaction mix contained (per 300 μL) 15 μl 1 of 1 M Tris Cl, 1.8 μL of 1 M sodium arsenate, 0.6 μL of 1 mM PLP, 1.5 μL of 0.1 M DTT, 54 uL of 1 M NaCl, 60 μl of | | | |
| # 0.3 M serine, 4.8 μl of 0.1 M NAD+, pure Salmonella TS, glyceraldehyde phosphate dehydrogenase (from yeast), and 100 μM IGP. | | | |
| # Inhibitors were tested at a maximum concentration of 100 μM. | | | |

**first active compound discovered

### Example 4

This example describes partial purification of endogenous plant TS and use thereof in an assay of TSβ assay.

Assay for Testing Inhibition of Tryptophan Synthase (TSβ-Reaction)

TS activity was measured in plant extracts by assaying TSβ activity. TSα activity could not be measured in plant extracts because other plant enzymes would degrade the substrate of the TSα reaction, IGP. Tryptophan synthase was assayed (i) in crude homogenates from plant tissues or (ii) as partially purified ammonium sulfate fractions from plant homogenates.

The assay was conducted in microfuge tubes by the TSβ reaction (indole+L-serine→L-tryptophan+H2O). 100 μL of extract was mixed with 150 μL of 0.4 mM indole, 80 mM serine, 0.03 mM PLP, 0.1 M Tris-Cl buffer pH 7.8, containing 7.5 μL of saturated NaCl. The mixture was incubated at 21° C. for increasing time intervals, from 10 min to several hours. The reaction was terminated by adding 25 μL 1 N NaOH, then 1 mL toluene with mixing, and then centifuging in a microfuge 2 min at 10,000×G to partition remaining indole into the toluene phase and away from the enzyme. Remaining indole was subsequently partitioned into the indole reagent phase and reacted with dimethylaminobenzaldehyde: 500 μL of the toluene layer from the microfuge tubes was mixed with 1 ml of the indole reagent in another tube and allowed to separate for 20 min, then the lower layer was carefully pipetted into a cuvette and its absorbance measured at 540 nm. This part of the assay was conducted as known in the art.

A unique microtiter plate method was also developed to streamline the partitioning steps and data collection. First, the TSβ reaction was performed as above in microfuge tubes. Then, after incubation and separation of indole from the assay solution, 150 μl of the indole-containing toluene phase was transferred to a polypropylene microtiter plate (any solvent resistant microtiter plate may be used) and 100 μl of the dimethylaminobenzaldehyde reagent was added. The plate was gently agitated. One drop of mineral oil was added to overlay the existing two liquid layers (thus resulting in three layers per well). The plates were centrifuged at a low speed, if necessary to flatten the horizontal surfaces of the middle phase. The lower reagent layer and the mineral oil should be separated by the toluene layer. The plate was covered by a mylar sheet (to protect the plate reader and avoid evaporation) and absorbance was monitored on the plate reader at 535 nm. The units used to express the results were nmol of indole reacted per hour per gram fresh weight of tissue, or nmol/hr/mg protein with protein assayed by the method of Bradford (Bradford, M., Anal. Biochem. 72,248 (1976)) using the commercial reagent from Bio-Rad Laboratories, Hercules, Calif.

Partial Purification of TS from a Higher Plant

TS was partially purified from spinach for use in the TSβ assay. The greatest degree of purification was achieved by homogenizing the tissue, preparing the 30-50% ammonium sulfate fraction and freezing it, thawing it, and applying the dissolved precipitate to an FPLC column (Waters SW300, Waters Corporation, Franklin, Mass.) to separate the TS activity (measured as TSβ) from the bulk of the protein. The yield was 34% with 180 fold purification. A similar method was used for maize TS. Subsequent chromatography on MonoQ with elution by NaCl improved the purity but led to a reduction in yield by partially removing TSα subunits from the holoenzyme. Because of the low yield of the assay of partially purified plant TS, endogenous enzyme was measured in crude extracts or in enzyme preparations involving one or two purification steps. As described later in Example 5, production of relatively pure plant TS was to require the use of transformed organisms.

Plant tissue to be used in the above TSβ assay was prepared as follows. Two grams of plant tissue were homogenized with a mortar and pestle in liquid nitrogen, then transferred to a second mortar and homogenized further in 0.1 mM PLP, 5 mM EDTA, 10 mM β-mercaptomethanol, 1 mM PMSF, and 50 mM KCl (total volume 10 ml), and centrifuged 20 min at 25,000×G. This was the crude homogenate. Ammonium sulfate was added to the supernatant to about 30% of saturation and the precipitate was removed by centrifugation. Ammonium sulfate was then added to the resulting supernatant to about 50% of saturation. The second precipitate was collected by centrifugation and dissolved in the assay solution described above to initiate the TSβ assay. Alternatively, the precipitate was frozen for further purification at a later time.

Alternatively, a single precipitation by ammonium sulfate at 80% of saturation was used to precipitate TS. Frozen pellets were washed once with the last solution, then resuspended in 0.5 ml homogenizing buffer per original gram fresh weight for assay. Dihydrotryptophan was used as a control. The TSβ activity is known to be inhibited by dihydrotryptophan.

### Example 5

This example shows production of active recombinant plant TSα subunit by over expression in E. coli. The methods and materials used in these experiments are described below.

Plant TSα Expression Vector Construction

To obtain large quantities (μg-mg) of active purified plant TS for analyses of inhibitors and modified TS genes, an E. coli based production system was developed. Three plasmids for expression of Arabidopsis TSα gene in E. coli were constructed. The plasmids were engineered to express the TSα coding sequence including: (i) a complete transit sequence (pAC757), (ii) a partial transit sequence (pAC758), and (iii) only a mature protein sequence (i.e., without the transit sequence) (pAC759).

The 5-prime PCR primer used to amplify a gene fragment coding for a TSα with a complete transit sequence (for pAC757 construction) contained the sequence 5′-GGGTTGGATCCATGGCGATTGCTT-3′. For a TSα construct with a partial transit sequence (pAC758), the 5-prime primer contained the sequence 5′-GATTCGGATCCATGGCTTCTCTCT-3′. For amplification of a gene fragment encoding only the putative mature TSα protein, the 5-prime primer contained the sequence 5′-AACAAGGATCCGTAGCATTCATACC-3′. The 3-prime PCR primer for each amplification contained the sequence 5′-TATCGATTTCGAACCCGGGTACCGA-3′. Each 5-prime primer was designed to contain a Bam HI restriction site, and the 3-prime primer was designed to contain an Eco RI site. The Arabidopsis TSα gene was used as a template. Each PCR-generated fragment was first cloned into the TA cloning vector (available from Invitrogen (Carlsbad, Calif.), and then subcloned in frame into the pGEX-2T vector (available from Pharmacia (Piscataway, N.J.). The completed expression vectors were transformed into the E. coli strain DH5α.

Plant TSα Purification from E. Coli Cultures

A 50 mL overnight culture of E. coli (DH5α) transformed with pAC753, pAC754, or pAC755 was used to inoculate 1 L of Luria Broth containing 50 μg/mL ampicillin and a 1: 1,000 dilution of sterile antifoam A. The culture was incubated at 37° C. with shaking for 4 hours. Protein expression was induced by the addition of IPTG to 1 mM (0.238 g/L) and the cells were cultured for additional 2.5 hours. Cells were harvested by centrifugation (5,000 rpm for 10 min in a Beckman JA-10 rotor) and immediately frozen and stored at −20° C. Frozen pellets were resuspended in 10 mL of MTPBS (150 mM NaCl, 16 mM Na₂HPO₄4 mM NaH₂PO₄, pH 7.3). Triton X-100 was added to final concentration of 1% and lysozyme was added to a final concentration of 100 μg/mL. The slurry was incubated at 30° C. for 15 min. Viscosity was reduced by mild sonication. The sample was centrifuged at 10,000 rpm for 10 min at 4° C. in a Beckman JA-20 rotor.

After lysis of the cells and centrifugation the supernatant was mixed with 2 mL of swollen glutathione agarose beads (sulfur linkage, Sigma Chemical Co., St. Louis, Mo.), 1 mL swollen solid beads, 1 mL buffer) and allowed to incubate with rocking for 45 minutes. The beads were settled by centrifugation (1,000 rpm table-top, centrifuge for 5 min) and the beads were washed with room temperature MTPBS. The washes were repeated 2 times. The washed beads were loaded onto a disposable column. The column was further washed MTPBS until the A₂₈₀ of eluent matched that of MTPBS. The fusion protein was eluted by competition with free glutathione (50 mM Tris.HCL pH 8.0 containing 5 mM reduced glutathione [available from Sigma] [final pH 7.5, freshly prepared]). All fractions with A₂₈₀ absorbance were pooled. SDS-PAGE analysis indicated a fusion protein of the expected molecular mass was expressed from each of the constructs. One mg of thrombin formulation (thrombin-bovine plasma thrombin, available from Sigma Catalog #T75 13) was added to the pool and the sample was dialyzed overnight at room temperature in 50 mM sodium citrate and 150 mM NaCl. SDS-PAGE indicated each fusion protein was cleaved into the respective GST and TSα proteins.

Plasmid pAC758 appeared to generate the greatest amount of TSα protein, however, based on the predicted molecular mass of TSα without a transit sequence the protein band may have been obscured by the GST protein band. No protein was detectable on gels for the cleaved TSα protein with a complete transit sequence however, this sample had TSα activity. The most protein and most activity was generated from pAC758.

Plant TSβ Expression Constructs

To obtain large quantities of active purified plant TS for analyses of inhibitors and modified TS genes an E. coli based production system was developed. Three plasmids for expression of the Arabidopsis TSβ coding sequence in E. coli were constructed. The plasmids were engineered to express TSβ with (i) a complete transit sequence (pAC753), (ii) a partial transit sequence (pAC754), or (iii) without the transit sequence, ie., expressing only the predicted mature TSβ protein (pAC755). Construction of pAC753 was initiated by PCR amplification of a TSβ gene fragment using primer 3 (5′-AACAGGGATCCGCAGCCTCAGGCA-3′) and primer 4 (5′-GTTTCTCGAATTCAAACATCAAGAT-3′) and the Arabidopsis TSβ gene as a template from Dr. G. R. Fink, MIT (M. B. Berlyn, et al., Proc. Natl. Acad. Sci. 86:4604-4608, June 1989). To generate a fragment containing TSβ coding sequence including a partial transit sequence (pAC754), primer 2 (5′-TCGTCTGGATCCAAGTCATCATCCT-3′) and primer 4 were used. To generate a fragment encoding a mature TSβ protein without the transit sequence, primer 1 (5′-ACCCGGATCCTTCGGTCGGTTT-3′) and primer 4 were used. Each 5-prime primer was designed to contain a Bam HI restriction site, and the 3-prime primer was designed to contain an Eco RI site. These restriction sites were used to clone the PCR fragments into the pGEX-2T E. coli expression vector (Pharmacia) in order to express a glutathione transferase/TSβ gene fusion protein. Each PCR amplified fragment was initially cloned into the Invitrogen TA cloning vector, and then subcloned to the pGEX-2T vector. The completed construct was transformed into the E. coli strain DHα.

The plasmids were constructed to include a 5 amino acid thrombin recognition site in order to be able to cleave the glutathione transferase (GST) protein from the TSβ protein. The protease cleavage resulted in two extra residues, Gly-Ser, on the N-terminal end of the TSβ protein. Each of the above vectors expressed the expected fusion protein, as well as the expected GST and TSβ proteins after thrombin treatment as confirmed on an SDS-PAGE gel.

Plant TSβ Purification from E.coli Cultures

A 50 mL overnight culture of E. coli (DH5α) transformed with pAC753, pAC754, or pAC755 was used to inoculate 1 L of Luria Broth containing 50 μg/mL ampicillin and a 1: 1,000 dilution of sterile antifoam A. The culture was incubated at 37° C. with shaking for 4 hours. Protein expression was induced by the addition of IPTG to 1 mM (0.238 g/L) and the cells were cultured for additional 2.5 hours. Cells were harvested by centrifugation (5,000 rpm for 10 min in a Beckman JA-10 rotor) and immediately frozen and stored at −20° C. Frozen pellets were resuspended in 10 mL of MTPBS (150 mM NaCl, 16 mM Na₂HPO_{4,}4mM NaH₂PO₄, pH 7.3). Triton X-100 was added to final concentrating of 1% and lysozyme was added to a final concentration of 100 μg/mL. The slurry was incubated at 30° C. for 15 min. Viscosity was reduced by mild sonication. The sample was centrifuged at 10,000 rpm for 10 min at 4° C. in a Beckman JA-20 rotor.

To purify the GST/TSβ fusion protein the supernatant was warmed to room temperature and mixed with a 1 mL slurry (0.5 mL swollen solid beads, 0.5 mL buffer) of glutathione agarose (sulfur linkage, available from Sigma Chemicals Co., St. Louis, Mo.) equilibrated with MTPBS. The sample was slowly mixed and incubated for 10 min. The beads were pelleted by centrifugation in a table top centrifuge by raising the rpms to 1500 and immediately shutting off the centrifuge. The supernatant was discarded and the beads were washed with 5 mL MTPBS and re-pelleted. The wash step was repeated 4 times. The fusion protein was eluted by addition of 0.5 mL 50 mM Tris-HCl (pH 8.0) containing 5 mM reduced glutathione (Sigma) (final pH 7.5, freshly prepared). The beads were again pelleted by low speed centrifugation and the supernatant was collected. The elution step was repeated an additional 2 times. The supernatants were filtered to remove any residual glutathione agarose beads. The GST/TSβ fusion protein was cleaved by addition of 0.5 mg of thrombin formulation (contains thrombin and buffer salts, Sigma Cat #T7513). The sample was then dialyzed against 2 L of 50 mM citrate, 150 mM NaCl, pH 6.5 overnight.

Plant TS Assay Using TSα and TSβ Expressed in E. coli

The plant TS proteins were expressed as fusion proteins with glutathione transferase (GST) to facilitate purification. After purification, the GST protein was cleaved off with thrombin as described above before the plant TS assays were performed. After thrombin cleavage, both TSα and TSβ-subunit proteins retained a Gly-Ser residue on the N-terminal of the protein in addition to the TS sequence. About 5 μg protein per assay for TSα and about 10 μg protein per assay for TSβ were used.

The TSα enzyme assay was conducted as described in Example 3 for Salmonella TSα. The results of the TSα enzyme activity are represented in Table 4.

TABLE 4

| | TSα activity, | TSα activity, |
|---|---|---|
| | relative | % of |
| Plasmid carried by the E. coli | Vmax | maximum |
| strain producing the extract | mOD/min | activity |
| pAC 757 (5 μg total protein) | 0.029 | <1 |
| pAC 758 (5 μg) | 0.025 | <1 |
| pAC 759 (5 μg) | 0.002 | <1 |
| pAC 757 (1.5 μg) + pAC 755 (3 μg)* | 0.959 | 17.7 |
| pAC 758 (1.5 μg) + pAC 755 (3 μg)* | 5.419** | 100 |
| pAC 759 (1.5 μg) + pAC 755 (3 μg)* | 0.066 | 1.2 |

*The TSα sample (cleaved fusion protein) was added to the reaction mix prior to addition of TSβ sample.
**This approached the limits of the assay.

The results in Table 4 indicate that the TSα protein expressed in E. coli is active. However, the TSα protein was fully active only in the presence of TSβ protein.

The TSβ assay was conducted as described in Example 4. The results of the assay are represented in Table 5.

TABLE 5

| | | | | | TSβ |
|---|---|---|---|---|---|
| | | | Indole | | activity |
| | Extract | Assay | converted, nmol | TSβ activity | mmole/ |
| Construct | volume | time | per assay | nmole/hr/ml | hr/mg |
| pAC755 | 100 μl | 18 hr | −0.4 | inactive | inactive |
| pAC754 | 5 μl | 1 hr | 10 | 2008 | 7.6 |
| pAC753 | 5 μl | 1 hr | 45.5 | 7899 | 11.1 |

Referring to Table 5, two of the constructs, pAC753 and pAC754, had very high TSβ activity, much greater than could be obtained using endogenous plant extracts, for example from spinach or maize. The TSβ without a leader sequence was inactive. However, the TSβ protein without a transit sequence was able to activate the TSα-subunit activity (see Table 4).

These data are consistent with the results obtained from the complementation experiments using E. coli mutants lacking tryptophan synthase activity, which experiments are described in Example 6. Referring to example 6, the mature Arabidopsis TSβ gene without a leader sequence was not able to complement E. coli. However, the TSβ gene expressing a complete transit sequence was able to complement the mutation.

### Example 6

The following experiments establish that the function of the plant TSβ subunit is conserved in comparison to the E. coli enzyme. The ability of the plant enzyme to complement the growth of an E. coli mutant strain that cannot grow without tryptophan supplementation as tested.

The E. coli mutant strain used contains a mutation in the endogenous enzyme gene. The strains EC972 (met⁻arg⁻trpB202) and NK7402 (trpB83::tn10) were obtained from the ATCC stock center. Strains W3110 trpA33 and W3110 tnA2 trpB9578 were a gift from Charles Yanofsky, Stanford University (Radwanski, E. R. et al., Mol. Gen. Genet. 248:657-667, 1995). All complementation tests were performed on M9 medium. The media was supplemented with both methionine and arginine for tests of EC972 transformants.

Plasmid pB1907, a gift from Dr. G. R. Fink MIT (M. B. Berlyn, R. L. Last, G. R. Fink; Proc. Natl. Acad. Sci. USA, 86:4604-4608, June 1989), contains the Arabidopsis TRPB gene encoding the TSβ subunit on a 2.1 kb EcoRI fragment. The EcoRI fragment was altered by including an NcoI site (CCATGG) surrounding the ATG start codon. The fragment was cloned into the E.coli expression vector pKK233-2 (available from Pharmacia, Piscataway, N.J.) by digesting with NcoI (5′ end of the gene) and Hind m (polylinker at 3′ end of gene) to create identical, independently isolated plasmids pAC502 and pAC505. The expression vector pKK233-2 contains the tac promoter and the rrnB ribosomal terminator.

The Arabidopsis TRPB sequence flanked by the pKK233-2 promoter and terminator was subcloned into the vector pACYC184 (New England Biolabs, Beverly, Mass.). First, both pKK233-2and pACYC184 plasmids were digested with Sca I and Eco RI in order to subclone the promoter terminator region into pACYC184 and create identical, independently isolated plasmids-pAC510 and pAC511. The fragment containing the Arabidopsis TRPB sequence was obtained from plasmid pAC502 by digesting it completely with HindIII and partially with NcoI. This resulting fragment was cloned into pAC510, which pAC510 was completely digested with NcoI and partially with HindIII to create identical, independently isolated plasmids pAC515 and pAC516.

Two independently isolated clones, pAC502 and pAC504, were transformed into E. coli strain EC972. This strain requires tryptophan supplementation for growth due to a mutation in the endogenous trpB gene. Transformants expressing the Arabidopsis TSβ were tested for their ability to grow on (i) unsupplemented minimal medium or (ii) minimal medium supplemented with indole, the substrate of TSβ subunit. The results of these tests are represented in Table 6.

TABLE 6

| STRAIN | LB | M9* | M9* | M9* + Indole | M9* + Tryptophan |
|---|---|---|---|---|---|
| EC972 | + | − | − | − | + |
| EC972 | + | ND | + | + | + |
| (pAC502) | | | | | |
| EC972 | + | ND | + | + | + |
| (pAC504) | | | | | |

ND = not determined
*M9 minimal media supplemented with methionine and arginine because strain EC972 is met⁻arg⁻

E. coli transformants expressing the Arabidopsis enzyme were able to grow on both the minimal medium and the minimal medium supplemented with indole, indicating that the plant enzyme is functional in E. coli.

This result was confirmed when the fragment containing the tac promoter, Arabidopsis TRPB gene and rrnB terminator were subcloned from plasmid pKK233-2 into plasmid. pACYC184. The resulting pAC515 and pAC516 plasmids were transformed into both W3110 tna2 trpB9578 (phenotype trpB⁻) and NK7402 trpB83::tn10 (phenotype trpA⁻trpB⁻). Five independent transformants carrying either pAC515 or pAC516 were plated onto (i) minimal media, (ii) minimal media supplemented with indole or (iii) minimal media supplemented with tryptophan. W3110 trpA33. (phenotype trpA⁻) and W3110 tnaA2 trpB9578 (phenotype trpB⁻) were patched as controls. The results of this complementation test are shown in Table 7.

TABLE 7

| STRAIN | LB | M9 | M9 + Indole | M9 + Tryptophan |
|---|---|---|---|---|
| W3110 trpB¹ (pAC515) | + | + | + | + |
| W3110 trpB (pAC516) | + | + | + | + |
| NK7402² (pAC515) | + | − | +/− | + |
| NK7402 (pAC516) | + | − | +/− | + |
| W3110 trpA33 | ND | − | + | + |
| W3110 trpB | ND | − | − | + |

¹W3110 trpB = W3110 tna2 trpB9578. Phenotype is trpB⁻.
²NK7402 trpB83::tn10. Phenotype is trpA⁻ trpB⁻.
ND—not determined

The Arabidopsis TSβ subunit was able to complement the growth of a strain carrying a mutation in the E coli trpB gene, and was able to complement the growth of an E. coli strain carrying mutations in both trpA and trpB when the media was supplemented with indole.

High Throughput Inhibitor Screening Method

The complementation of the E. coli strains deficient in endogenous TS activity by expression of plant enzymes enables screening for inhibitors of plant TS in a high throughput manner. Screens can be run in duplicate plates of minimal media with or without supplementation with tryptophan. A lawn of the E. coli strains may be incorporated in the plates, and the plates then spotted in a replicated pattern with chemical compounds to be tested. Compounds that produce a zone of clearing in the medium without tryptophan but have smaller or no zone of clearing in the medium supplemented with tryptophan are indicative of inhibitors of the tryptophan biosynthetic pathway. Compound identified in this manner may be further analyzed by enzyme assays or other methods described herein or known to persons of skill in the art. The advantage of performing the screenining in a bacterium is that a high number of compounds may be screened in a high throughput and automated manner.

The same E. coli strains complemented with the Arabidopsis TSα or the TSβ genes are used for identifying mutations that confer resistance to TS inhibitors in a high throughput manner. Such variant resistant genes are useful for conferring resistance to crops for TS inhibiting herbicides. The E. coli strains are mutagenized and plated on minimal M9 media containing the herbicide. Strains with plasmids harboring a resistant variant of the plant TS enzyme are recovered. The TS genes are sequenced to identify mutations. These resistance genes are transformed into crops.

### Example 7

This example demonstrates successful inhibition of Arabidopsis TS enzyme (produced recombinantly in E. coli as described in Example 4) with the inhibitors of the invention. Specifically, phenylthiophosphonic acid compounds were used. The TSα assay conditions were as described for Salmonella TSα in Example 3 except that recombinant plant proteins were used instead of the Salmonella enzyme. The results are represented in Table 8.

TABLE 8

| | TSα activity, | |
|---|---|---|
| Inhibitor | relative Vmax | TSα activity, |
| (82 uM) | mOD/min | % of control |
| control: pAC 758 (1.5 ug) + | 6.198** | 100 |
| pAC 755 (3 ug) with no inhibitor* | | |
| indolepropanol phosphate (standard) | 2.223 | 35.9 |
| Phosphonic acid, {4-[(5-bromo-2- | 0.238 | 16.1 |
| hydroxyphenyl)thio]-1-butenyl}- | | |
| Phosphonic acid, {4-[(o- | 0.909 | 14.7 |
| hydroxyphenyl)thio]-2-butenyl}- | | |
| Phosphonic acid, {4-[(2- | 0.168 | 2.7 |
| hydroxyphenyl) | | |
| thio]butyl}-, | | |
| benzoate (ester) | | |
| Phosphonic acid, {4-[(o- | 0.150 | 2.4 |
| hydroxyphenyl)sulfonyl]butyl}- | | |
| Phosphonic acid, {4-[(o- | 0.133 | 2.1 |
| hydroxyphenyl) | | |
| thio]butyl}-,aryl-butyrate (ester) | | |
| Phosphonic acid, {4-[(o- | 0.095 | 1.5 |
| hydroxyphenyl)sulfinyl]butyl}- | | |

*The E. coli pAC 758 (1.5 ug) cleavage proteins were added to the reaction mix prior to addition of the E. coli pAC 755 (3 ug) cleavage proteins.
**This approached the limits of the assay.

These results demonstrate that the compounds designed to inhibit the Salmonella enzyme also inhibit TS enzymes from higher plants. Accordingly, an assay containing a microbial TS enzyme may be used as a test system for identifying and assaying novel inhibitors of plant TS.

### Example 8

This example establishes that inhibitors identified using Salmonella TSα also inhibit the plant TS enzyme as using a TSβ assay. The enzyme from spinach was purified as described in Example 4.

Inhibitory compounds that were active on the Salmonella enzyme (measured in a TSα assay) were also active on the spinach enzyme (measured in a TSβ assay according to Example 4). In these experiments, the TSα activity was determined quantitatively, while the TSβ activity was determined qualitatively. The results are represented in Table 9.

TABLE 9

| | | TSβ |
|---|---|---|
| | TSα activity | (Spinacea |
| | (Salmonella | enzyme), |
| | enzyme) | relative |
| Compound | I₅₀, nM | activity |
| Phosphonic acid, | 130 | + |
| {4-[(o- | | |
| hydroxyphenyl)thio] | | |
| butyl}- | | |
| Phosphonic acid, | 550 | +++++ |
| {4-[(o- | | |
| aminophenyl)thio]butyl}-, | | |
| with | | |
| cyclohexylamine | | |
| (1:2) | | |
| Phosphonic acid,- | 1000 | +++++++* |
| {4-[(2-amino-p- | | |
| tolyl)thio]butyl}- | | |

*the increase in the number of “+” corresponds to the increase of inhibition

### Example 9

The following results establish that the inhibitors of the invention are also inhibitors under in vivo conditions.

Previous examples demonstrate that the compounds of the invention are potent inhibitors of both microbial and plant TS enzymes in vitro. However, these compounds could have had a different mechanism of action in vivo. It was therefore important to demonstrate that the herbicidal effects of the compounds was due to blocking tryptophan biosynthesis. Reversal assays (also known as rescue, prevention or complementation) described below demonstrate that the expected mechanism of action (i.e., blocking of tryptophan biosynthesis) was in fact occurring in plants.

Reversal of Herbicidal Activity of TS Inhibitors in Arabidopsis

Reversal of herbicidal symptoms by metabolites, products of biosynthetic pathways, or other compounds can indicate the mechanism of action of herbicidal compounds.

In this experiment, TS inhibitors were tested on Arabidopsis thaliana grown Murashige minimal organics medium, (obtained from Life Technologies, Grand Island, N.Y.), containing 0.7% agar. Compounds were tested at different concentrations to assess their herbicidal activity. The results demonstrating the reversal of herbicidal activity of the TS inhibitors With tryptophan are represented in Table 10.

TABLE 10

| | Concentration of the herbicide | | | | | | | |
|---|---|---|---|---|---|---|---|---|
| | (mM) | | | | | | | |
| Treatment | 1000 | 500 | 250 | 125 | 63 | 31 | 16 | 7.8 |
| Phosphonic | 6C | 6C | 6C | 6C | 6C | 5C | 5C | 5C |
| acid, | | | | | | | | |
| {4-[(o- | | | | | | | | |
| hydroxyphenyl) | | | | | | | | |
| thio]- | | | | | | | | |
| 1-butenyl}- | | | | | | | | |
| Phosphonic | | | | | | | | |
| acid, | 0 | 0 | 0 | 0 | 0 | 0 | 0 | 0 |
| {4-[(o- | | | | | | | | |
| hydroxyphenyl) | | | | | | | | |
| thio]- | | | | | | | | |
| 1-butenyl}- + | | | | | | | | |
| 100 μM | | | | | | | | |
| Trp | | | | | | | | |
| Phosphonic | | | | | | | | |
| acid, | 8 | 7Y | 7Y | 6 | 6 | 6 | 6 | 5 |
| {4-[(o- | | | | | | | | |
| hydroxyphenyl) | | | | | | | | |
| sulfinyl] | | | | | | | | |
| butyl}-, | | | | | | | | |
| with | | | | | | | | |
| cyclohex- | | | | | | | | |
| ylamine | | | | | | | | |
| (1:2) | | | | | | | | |
| Phosphonic | 5 | 5 | 5 | 5 | 5 | 3 | 0 | 0 |
| acid, | | | | | | | | |
| {4-[(o- | | | | | | | | |
| hydroxyphenyl) | | | | | | | | |
| sulfinyl] | | | | | | | | |
| butyl}-, | | | | | | | | |
| with | | | | | | | | |
| cyclohex- | | | | | | | | |
| ylamine | | | | | | | | |
| (1:2) + 100 μM | | | | | | | | |
| Trp | | | | | | | | |
| Phosphonic | 7 | 7 | 7 | 7 | 7 | 6 | 6 | 5 |
| acid, | | | | | | | | |
| {4-[(o- | | | | | | | | |
| hydroxyphenyl) | | | | | | | | |
| thio] | | | | | | | | |
| butyl}-, aryl- | | | | | | | | |
| butyrate (ester) | | | | | | | | |
| Phosphonic | 3 | 3 | 5 | 5 | 3 | 1 | 1 | 1 |
| acid, | | | | | | | | |
| {4-[(o- | | | | | | | | |
| hydroxyphenyl) | | | | | | | | |
| thio] | | | | | | | | |
| butyl}-, aryl- | | | | | | | | |
| butyrate | | | | | | | | |
| (ester) + | | | | | | | | |
| 100 μM | | | | | | | | |
| Trp | | | | | | | | |
| Phosphonic | 7 | 6 | 6 | 6 | 6 | 6 | 5 | 5 |
| acid, | | | | | | | | |
| {4-[(o- | | | | | | | | |
| hydroxyphenyl) | | | | | | | | |
| thio] | | | | | | | | |
| butyl}- | | | | | | | | |
| Phosphonic | 3 | 3 | 5 | 3 | 1 | 0 | | |
| acid, | | | | | | | | |
| {4-[(o- | | | | | | | | |
| hydroxyphenyl) | | | | | | | | |
| thio] | | | | | | | | |
| butyl}- + | | | | | | | | |
| 100 μM | | | | | | | | |
| Trp | | | | | | | | |

Ratings:
0 - no effect,
9 - total kill,
C - chlorotic seedlings 4-6 days after treatment

Referring to Table 10, TS inhibitors were herbicidal at a wide range of concentrations, causing severe stunting and chlorosis of the seedlings, that ultimately led to the death of the plants. These symptoms were completely prevented by the addition of L-tryptophan to the growth medium. Plants that were treated with the herbicides were dying, while the plants treated with the herbicides and L-tryptophan looked healthy and did not differ from untreated plants. Tryptophan was the only amino acid that was capable of complete reversal of herbicidal activity of these TS inhibitors. These results indicate that compounds that inhibit TS in vitro are herbicidal in vivo, and that the herbicidal activity in vivo is due solely to inhibition of tryptophan biosynthesis.

Accordingly, herbicidal compounds that inhibit TS can be identified using a reversal assay with tryptophan. This method can be used initially as a high throughput screening assay, or as a secondary assay to identify and confirm that tie mechanism of action of a particular inhibitor is due to inhibition of tryptophan biosynthesis.

### Example 10

The results of this experiment demonstrate that esters are more effective inhibitors in vivo that free acids analogs.

Plants possess esterase enzymes which remove ester groups from many xenobiotics, although de-esterification of a specific compound may occur more rapidly in some species than in others. Furthermore, variation in the basal molecular structure may influence the rate of de-esterification in an individual species. The following results indicate this effect on herbicidal injury to Arabidopsis, and explains why some esters may be less effective on TS under in vitro conditions than in vivo, in the greenhouse. The results are represented in Table 11.

TABLE 11
Concentration of the herbicide (μM)

| Treatment | 1000 | 500 | 250 | 125 | 63 | 31 | 16 | 7.8 |
|---|---|---|---|---|---|---|---|---|
| Phosphonic acid, | 4 | 4 | 4 | 3 | 3 | 2 | 1 | 1 |
| {4-[2-amino-5- | | | | | | | | |
| bromophenyl)thio] | | | | | | | | |
| butyl}-(acid) | | | | | | | | |
| Phosphonic acid, | 8 | 6 | 5 | 4 | 1 | 1 | 1 | 1 |
| {4-[2-amino-5- | | | | | | | | |
| bromophenyl)thio] | | | | | | | | |
| butyl}-, | | | | | | | | |
| diethyl ester | | | | | | | | |
| (ester) | | | | | | | | |
| Phosphonic acid, | 3 | 3 | 3 | 3 | 1 | 1 | 1 | 0 |
| {4-[(2-amino-5- | | | | | | | | |
| chlorophenyl)thio] | | | | | | | | |
| butyl}-(acid) | | | | | | | | |
| Phosphonic acid, | 7 | 6 | 5 | 3 | 1 | 1 | 0 | |
| {4-[(2-amino-5- | | | | | | | | |
| chlorophenyl)thio] | | | | | | | | |
| butyl}-, | | | | | | | | |
| diethyl ester | | | | | | | | |
| (ester) | | | | | | | | |
| Phosphonic acid, | 6C | 5C | 3C | 3C | 3C | 1C | 1 | 0 |
| {4-[(o- | | | | | | | | |
| hydroxyphenyl)thio] | | | | | | | | |
| butyl}- | | | | | | | | |
| (acid) | | | | | | | | |
| Phosphonic acid, | 9 | 8 | 7 | 6 | 5 | 3 | 1 | 0 |
| {4-[(o- | | | | | | | | |
| hydroxyphenyl)thio] | | | | | | | | |
| butyl}-, | | | | | | | | |
| diethyl ester | | | | | | | | |
| (ester) | | | | | | | | |

Ratings:
0 - no effect,
9 - total kill,
C - chlorotic seedlings

Accordingly, in practice, compounds which are herbicidal inhibitors of TS may be routinely synthesized as diesters and certain salts to improve the compound delivery to the target site within the plant.

### Example 11

This example describes the reversal assay in Synechocystis.

End-Product Reversal in Synechocystis

Synechocystis is a unicellular green organism that is actually a photosynthetic bacterium, with a photosynthetic system very similar to that of higher plant chloroplasts. Culture growth of Synechocystis could be inhibited by a compound of the present invention, and the growth inhibition could be prevented in the presence of tryptophan.

Tryptophan completely reversed the growth inhibitory effects of the benzoate ester of 4-[(2-hydroxyphenyl)thio]butyl-phosphonic acid on the cyanobacterium Synechocystis PCC 6803.

TABLE 12

| | Culture density as A₄₂₀* | | | | | |
|---|---|---|---|---|---|---|
| Inhibitor, | no | | +tryptophan, | | +tryptophan, | |
| μM | tryptophan | (%) | 31 μM | (%) | 62 μM | (%) |
| 0 | 0.534 | 100 | 0.642 | 120 | 0.599 | 112 |
| 62 | 0.449 | 84 | 0.726 | 136 | 0.704 | 132 |
| 125 | 0.040 | 7 | 0.622 | 116 | 0.544 | 102 |
| *The assay was conducted in liquid medium in microtiter plates with the inhibitor added at time zero (culture dilution) and the activity measured four days thereafter. | | | | | | |
| # Greater concentrations of the inhibitor or of tryptophan were inhibitory. | | | | | | |

### Example 12

A number of factors determine whether a specific target within a plant is a good herbicide target. These factors include the importance of the target and its function in the health of the plant, the flow of metabolites in the pathway in which the target is involved, the mechanism by which a plant is compromised by inhibition of the target, the localization of the target enzyme, and the abundance of the target in the target species. To assess TS as a herbicide target, TS targeted herbicides and the TS enzyme in crop and weed species were characterized. The results are described in this and the following examples.

TS inhibitors Cause Early Damage to Upper Shoot Tissues

Herbicidal compounds of the invention were examined for their herbicidal effects by observing symptoms on postemergence treated plants. Injury symptoms suggested that the young shoots were most sensitive to these herbicides.

Early injury symptoms caused by the TS-inhibiting herbicides of the invention are represented in Table 13. Symptoms and species effects are represented through the herbicidal activity {4-[(o-hydroxyphenyl)thio]butyl}-phosphonic acid, applied at 4 or 8 kg/ha.

TABLE 13

| Symptoms 6 DAT* | Species effects, 13 DAT** |
|---|---|
| leaf yellowing: mustard, hemp | mustard: little growth between 6 and |
| sesbania, | 13 dat |
| leaf mottling: mustard, soybean, | lambsquarters: 40% height reduction, |
| lambsquarters, pigweed, bindweed, | green |
| morningglory | |
| shoot tip yellowing: hemp sesbania | pigweed: 25% height reduction |
| tip necrosis: hemp sesbania | bindweed: mottling, some necrosis, |
| | green cotyledon leaves |
| height reduction: lambs quarter, | hemp sesbania: no growth between 6 |
| soybean | and 13 dat, much reduced vigor |
| increased branching: soybean | soybean: shoot nearly dead except |
| | cotyledons dark green |
| | corn: unaffected |
| | green foxtail: stunted, yellowing, red |
| | tips of leaves |
| | velvetleaf: 50% height reduction |

*Symptoms are described 6 days after post emergence application of inhibitors
**Species effects are described 13 days after post emergence application

### Example 13

The results represented in this example establish that TS is concentrated in actively growing, developing plant tissues.

TS from ammonium sulfate precipitates prepared according to Example 4 was assayed using the TSβ reaction and the results were expressed as nmole indole used per hour per gram fresh weight or as nmol/h per mg tissue protein. Experiments using spinach, corn, and tomato demonstrated that the young, growing or developing tissues possess the greatest amounts of TS enzyme. This correlates well to the type of injury symptoms seen in a variety of plant species treated with TS-inhibiting herbicides. In contrast, stem and root tissue did not have measurable amounts of the enzyme. This correlates to the fact that higher plant genes for TS contain signal sequences that target the proteins to chloroplasts.

The results demonstrating that differentiating and growing tissues contained the highest TS activity in Spinacea oleracea are represented in Table 14. All tissues except the mature leaves were differentiating and/or growing tissues.

TABLE 14

| | | TS specific |
|---|---|---|
| | TS activity | activity, |
| | mg protein/ | nmol/hr/ |
| stage of development | g tissue | mg protein |
| young leaves (80 mg each) from 21d-old | 6.6 | 40.5 |
| plants with 8 leaves | | |
| mature leaves (670 mg each) from 35d-old | 4.8 | 15.3 |
| plants, not bolting | | |
| bolting plants, terminal meristems (290 mg | 8.0 | 28.2 |
| each) with no visible floral buds, most bracts | | |
| removed | | |
| flowering raceme (1¼ inch, 1 g each), buds | 5.0 | 39.2 |

Maize tissue cultures were another source of endogenous TS. The amount of activity recovered was dependent on the genotype and/or the state of the cultures. Partial purification produced enzyme that eluted identically as the spinach TS on the Waters SW300 column. TS activity from maize cultures was assayed by the β reaction.

The results demonstrating that differentiating cell cultures (type II callus) of maize had more TS activity than slow growing cell suspension cultures (late log phase) are represented in Table 15.

TABLE 15

| | | | TS specific |
|---|---|---|---|
| | | | activity, |
| | | TS activity, | nmoL/hr/ |
| Genotype | | nmol/hr/g | mg protein |
| A188 x B73 | type II callus, high auxin | 396 | 46 |
| black mexican | cell suspension, late log | 206 | 23 |
| sweet corn | phase | | |

TS from tomato (Lycopersicum esculentum) was used to compare TS levels to tissue age. The following plant material was used: mature plants with many mature tomatoes; flowering plants at the 10-leaf stage; and young seedlings 19 days old. Young growing tissue on vigorously developing plants had the greatest enzyme activity and specific activity. The specific activity was measured by the Bradford protein assay. The results demonstrating that high TS activity correlates to tissue that is active growing and/or differentiating in Lycopersicum esculentum are represented in Table 16.

TABLE 16

| | | TS activity, | TS specific |
|---|---|---|---|
| | mg protein/ | nmol/h | activity, |
| Growth stage and | g fresh | per gram | nmol/h/mg |
| tissue | weight | fresh wt | protein |
| small leaves | 23.7 | 4.1 | 0.17 |
| mature plant | | | |
| oldest green leaves | 12.7 | 5.1 | 0.40 |
| mature plant | | | |
| second leaf from top | 24.3 | 139 | 5.72 |
| flowering, no fruit | | | |
| oldest leaf | 7.5 | 3.1 | 0.41 |
| flowering, no fruit | | | |
| flowers and buds | 10.2 | 7.4 | 0.73 |
| flowering, no fruit | | | |
| entire shoot of young | 21.0 | 22.0 | 1.0 |
| seedling | | | |

Rapidly growing “sink” tissues have much higher TS levels than slow or non-growing “source” tissues. “Sink” tissues exhibit a net gain of certain nutrients and organic metabolites with time, while “source” tissues are reduced in those nutrients. Young, rapidly expanding leaves on non-flowering plants with 5 leaves (sink tissue) had higher TS activity than did leaves at the base of the first flowering truss of flowering, 7-leaf plants (source tissue).

The results demonstrating that “sink” leaf tissue had greater TS activity than “source” leaf tissue in tomato in tomato are represented in Table 17.

TABLE 17

| | TS activity, nmol/h | TS specific |
|---|---|---|
| | per gram fresh wt | activity, |
| Growth stage and tissue | of plant tissue | nmol/h/mg protein |
| Non-flowering plant, young | 80.3 | 3.8 |
| leaves (sink tissue) | | |
| First truss, leaf at base of | <1 | <0.1 |
| truss (source tissue) | | |

Shoot tips on plants of all ages had the greatest TS activity. Only after fruiting did the TS activity decline at the shoot tip. Thus TS inhibitors would be most effective applied to or reaching the growing shoot tips of plants.

The results demonstrating that shoot tips from tomato plants of all ages have greatest TS activity prior to fruiting are represented in Table 18.

TABLE 18

| | | TS activity in the | TS specific |
|---|---|---|---|
| | | shoot tip, nmol/h | activity in the |
| Days after | Growth stage of the | per gram fresh wt | shoot tip, |
| planting | tomato plant | of plant tissue | nmol/h/mg protein |
| 20 | two full leaves | 194 | 9.3 |
| | plus tip, unbranched | | |
| 27 | three full leaves, | 163 | 7.2 |
| | unbranched | | |
| 36 | seven full leaves, | 138 | 7.3 |
| | unbranched | | |
| 41 | eight full leaves, 1 | 177 | 9.0 |
| | branch | | |
| 48 | 12 full leaves, 2 | 175 | 7.4 |
| | branches, flowering, | | |
| | not fruiting | | |
| 69 | numerous leaves, 5 | 79 | 3.3 |
| | branches, fruiting | | |

*A full leaf was a leaf with at least 5 leaflets expanded. The largest leaf of each shoot tip was about 8 cm along the rachis.

The results demonstrating that tissues below the shoot tip have little TS activity are represented in Table 19. Greenhouse tomato seedlings were extracted 22 days after planting. The root tissues and stem tissues below the shoot tip had no measurable TS activity.

TABLE 19

| | | TS activity, | |
|---|---|---|---|
| | | nmol/h | |
| | | per gram | TS specific |
| | mg protein/ | fresh wt | activity, |
| | g fresh | of plant | nmol/h/mg |
| Tissue | weight | tissue | protein |
| shoot tip | 24.9 | 135 | 5.63 |
| (leaves less than 3 cm) | | | |
| stem below tip | 2.7 | <1 | <0.1 |
| tender roots* | 1.8 | <1 | <0.1 |

*the root tips may have been damaged when the soil was removed

The results representing that small leaves at the tops of tomato plants of different ages had greater TS activity than larger leaves near the tops of tomato plants of different ages are shown in Table 20. There was a logarithmic correlation of TS activity to fresh weights of the leaves (regression correlation of 0.74), with the maximum activity at 0.1 to 0.6 g fresh weight per leaf and less than 10% of that activity at 4 g per leaf or higher (Table 20).

TABLE 20

| Leaf fresh weight, g | TSβ activity, nmol/h/g |
|---|---|
| 0.12 | 186 |
| 0.64 | 232 |
| 1.26 | 119 |
| 2.13 | 65 |
| 3.85 | 9 |
| 4.10 | 2 |

*Leaves were removed from plants that were planted 13 d, 27 d, 40 d, and 81 days previously, and the TS levels were measured using the TSβ reaction

### Example 14

The results reported in this example establish levels of TS activity in several weeds.

TS is not an abundant enzyme, and in the examples of tomato and spinach above, even the highest levels of TSβ activity were generally less than 200 nmol/h/g fresh weight of plant tissue. Most seedlings had even lower TS activity than tomato or spinach. The TSβ activity was assayed as decribed in Example 4. Weed species were planted into a synthetic potting mix in the greenhouse for either 2 weeks (annual weeds from seeds) or 4 weeks (perennial weed species). The plants were not treated by herbicides, but weed seedlings used for the experiment were of a size equivalent to that for early post emergence application of herbicides.

The results demonstrating a very low TS activity level in some key weeds are represented in Table 21. Many weeds had TS activity that was too low to measure. Thus TS is a good herbicide target in the sense that the amount of active enzyme is already low. When ammonium sulfate precipitates (25 to 60%) (prepared according to Example 4) were assayed for TSβ activity, only Sinapis arvensis and Elytrigia repens had measurable activity.

TABLE 21

| | | TSβ specific |
|---|---|---|
| | TSβ activity, | activity, nmol/h/ |
| Species* | nmol/h/g | mg protein |
| Cyperus rotundus, Calystegia sepium, | nil | nil |
| Digitaria sanguinalis, Setaria viridis, | | |
| Ipomoea hederaceae, Avena fatua, | | |
| Abutilon theofrasti, Ambrosia | | |
| artemisiifolia, Sesbania exaltata | | |
| Sinapis arvensis | 32.5 | 2.0 |
| Elytrigia repens | 7.1 | 0.8 |
| Spinacea oleracea (for comparison) | 118.2 | 11.4 |

*Annual weed species (upper shoots) were extracted 2 weeks after planting and the perennials 4 weeks after planting

### Example 15

This example establishes that TS is present in maize seedlings grown in hydroponics.

The results demonstrating TS activity as distributed in young maize seedlings are represented in Table 22.

Maize was extracted for TS activity in 5 day-old seedlings grown in hydroponics to avoid soil particles attaching to the roots. Hydroponic conditions were established by germinating the seedlings in moist paper towels, then placing only the roots of individual seedlings in a 2 oz glass jar containing a suitable, dilute, mineral solution. Tissue samples were evaluated using the TSβ assay. Before the assay was conducted, the extracts were passed over a DP10 sizing column. The intercalary meristem zone was that which contained the lower whorl leaf tissue, and included the shoot meristem.

TABLE 22

| | | TS activity, nmol/h | TS specific |
|---|---|---|---|
| | mg protein/ | per gram | activity, |
| | g fresh | fresh wt | nmol/h/mg |
| Tissue | weight | of plant tissue | protein |
| green leaf blade, 1^{st} | 10.1 | 170.4 | 16.8 |
| leaf | | | |
| intercalary meristem | 7.3 | 53.3 | 7.3 |
| zone | | | |
| root | 2.5 | 4.2 | 1.7 |

Referring to Table 22, the young leaf blade had more TS activity than the lowest part of the shoot whorl tissue or the root.

### Example 16

This example describes production of antibodies to plant tryptophan synthase β-subunit.

Antibodies to the tryptophan synthase β-subunit (TSβ) can be used to assess the location and level of expression of the enzyme in target tissues. It can also be used as an analytical reagent for expression of the protein in heterologous systems.

The TSβ subunit was expressed from pAC755, purified, and digested with thrombin as described in Example 5.

To the thrombin digested preparation (volume of 11 mL), a ⅕th volume of 5×SDS sample buffer (50% glycerol, SDS, bromophenol blue), and 1/10th volume of 1 M DTT were added. The sample was placed in a boiling water bath for 3 minutes and stored at 4° C. A 12.5% SDS PAGE preparative gel (Laemlli, 1.5 mm wide) was prepared and loaded with 2 mL of the SDS treated sample. Also loaded on the gel were 2 lanes of Bio-Rad prestained standards. The gel was electrophoresed at 40 mAmp through the stacking gel and 60 mAmp through the resolving gel. A portion of the gel containing a set of standards and the TSβ preparative portion of the gel was removed and stained with Coomasie Blue. The remainder of the gel was placed in a 1 M KCl solution. Proteins precipitating in the KCl-treated gel were visualized. The portion of the gel containing the TSβ protein were cut out and washed with distilled water to remove the KCl. The gel slice was stored at −20° C.

The gel slice containing the TSβ protein was placed in a conical tube and the tube was frozen on dry ice. A hole was pierced through the conical tube and the gel slice was lyophilized. The lyophilized sample was powdered by grinding with a glass rod. A sample of the lyophilized gel was weighed and run on an SDS-PAGE gel loaded with known amounts of BSA as standards. It was estimated that approximately 5.0 μg of TSβ protein was contained in each mg of lyophilized acrylamide gel. Approximately 10 μg of TSβ protein was suspended in 0.8 mL of RIBI MPL+TDM adjuvant. 0.2 mL of the sample was used to immunize mice intraperotoneally. After four immunizations, ascites was collected.

The antisera raised to Arabidopsis TS were able to recognize TSβ protein expressed in E. coli. The antisera to Arabidopsis TS were also tested against crude extracts of Arabidopsis. No signal was detected indicating that the TS protein is expressed at very low levels in plants. The low abundance of the protein can be advantageous for exploiting TS as a herbicide target.

### Example 17

In this example a high-resolution crystal structure of a Salmonella TS complexed with phosphonic acid, {4-[(2-amino-5-chlorophenyl)thio]butyl}-was obtained to study the details of the binding of the inhibitor of this invention using molecular modeling techniques. The studies have resulted in a better understanding of the critical features of substrate and inhibitor binding, which is critical for further design of improved inhibitors and herbicides.

Tryptophan Synthase was prepared as described above and co-crystallized with {4-[(2-amino-5-methoxyphenyl)thio]butyl}-phosphonic acid. The compound was prepared as described in the U.S. Pat. No. 5,635,449 to Langevine and Finn.

The protein-inhibitor complex was prepared by mixing (4-[(2-amino-5-methoxyphenyl)thio]butyl}-phosphonic acid, and TS to final concentrations of about 10 mg/mL and 5 mg/mL. Crystals of the complexes were grown under conditions as described above. The diffraction data were collected at 100K in 1° steps. The crystals exhibit symmetry of the space group C2 with one αβ pair in the asymmetric unit. Cell parameters were a=183.3 Å, b=59.5 Å, c=67.3 Å, alpha=gamma=90°, beta=94.78 For the refinement, at a cutoff of two times sigma (F>20F), 47362 unique reflections have been used from the resolution range between 29 Å and 2 Å, corresponding to a completeness of 96% total and 91% at the highest resolution. An iterative refinement protocol used a simulated annealing procedure to refine the structure and add 160 water molecules to a final R value of 0.21. The refinement protocol was very similar to the protocol described in the following example except that the all visualization and the placement of solvent, cofactor, and inhibitor molecules have been performed using the program Quanta (MSI).

The electron density of the final model of TS with bound phosphonic acid, {4-[(2-amino-5-methoxyphenyl)thio]butyl}-reveals the details of the phosphonate binding as discussed in the specification. It also revealed for the first time that αPhe212 has very unusual backbone dihedral angles, with the α-Carbon-Hydrogen bond pointing toward the phosphonate group and the phenyl ring system being placed above the ring system of the inhibitor, thus providing a T-shaped aromatic-aromatic interaction to the aryl ring of the inhibitor.

Electrostatic potential calculations used a Finite Element Poisson-Boltzman calcluation as implemented in the program DELPHI (MSI) and with a two step procedure and parameters as described in Bashford and Karplus, Biochemistrym 1990, 29, 10219. In this grid based numerical calculation, the solvent effect on the protein electrostatics is traeted implicitly. The area of the protein is treated at a dielectricity constant (εᵣ) of 4, while the outside (as defined by a Connolly surface calculation using a 1.4 Å probe radius) has assigned a εᵣ=78. The radius of ions was assumed to be larger than 2 Å.

In the first calculation of the Tsα-subunit with partial charges on each atom taken from the CVFF force field (MSI), a cubic grid of 100 Å edge length and 1 Å grid spacing, centered at αE49, was calculated setting the grid points at the cubus surface at zero. A focusing of the grid with 101 grid points spaced 0.25 Å apart to achieve a high-resolution grid around the center of interest was then calculated with the grid points for the outermost planes set to the values from the first calculation. The two values for the electrostatic potential of the protein (Pu) and the protein with αE49 protonated (Pp), and the corresponding pair of energy values for this amino acid in the same position and conformation but without the remainder of the protein in protonated (Ap) and unprotonated (Au) form have been calculated. Based on the difference between the electrostatic free energy of the protein protonated at αE49 (Pp-Pu) and the protonation of αE49 in solution (Ap-Au), the change of the pKa for αE49 was calculated to be about 8. This is in good agreement with an experimentally derived values of 7.5 (Yutani et al., J. Biol. Chem, 259:14076-81, 1984) and 8.5 (Sawada et al. Eur. J. Biochem, 189:667-613, 1990). Similar calculations revealed that Asp 60 is more acidic by about 1 pKa. This rather unusual pKa value for αE49 results from its position in a hydrophobic surrounding and the presence of αD60. The negative charge of this amino acid αD60 increases the energy of deprotonating αE49 since this creates a hydrophobic crevice deep within the protein with two close, uncompensated negative charges. This change in the pKa value for αE49 destabilizes the folded conformation of the enzyme. Introduction of a group that could form a salt bridge with αE49 would therefore free this potential energy in the form of binding energy. This would be a similar interaction to the one between the amino group of the inhibitor of the invention and αD60.

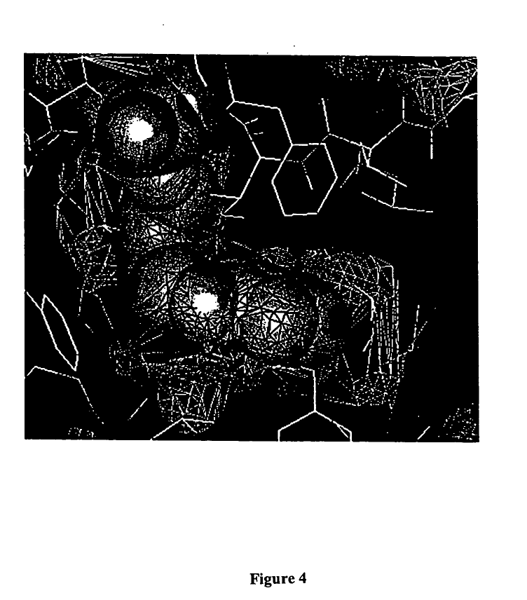

The electrostatic potential energy grid can also be used to visualize the interaction surface between the protein and the inhibitor, thus allowing the chemist to visualize details of the protein-inhibitor interaction. An example for such a display is given in FIG. 4.

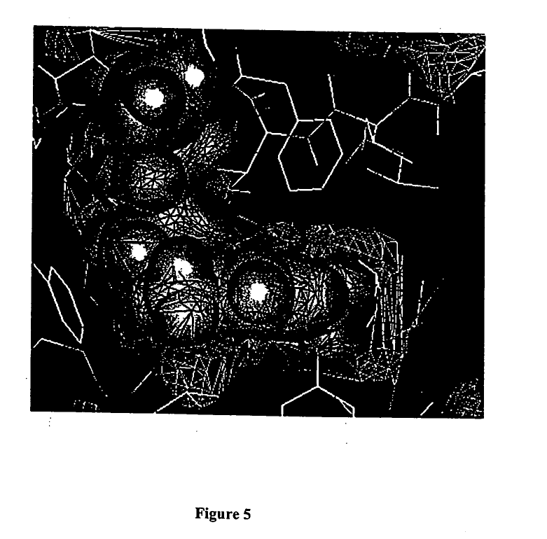

Such visualization, in particular, when used with stereo displaying facilities are of importance for the synthetic chemists to develop new ideas for chemical modifications. Most of the conceptual work for the synthesis program was based on the early access of crystallographic information. For example, the analysis of the conformation of the IPPP bound to αTS indicates an almost 90° angle between the plane of the indole and the linker (FIG. 4). In addition, the analysis shows that the indole part fills the available active site pocket rather poorly. Introduction of a sulfur as a linker and an elongation of the linker resulted in a series of inhibitors of much superior performance (FIG. 5).

### Example 18

The following example describes crystal structures of a Salmonella TS complexed with a series of phosphonate inhibitors of the invention.

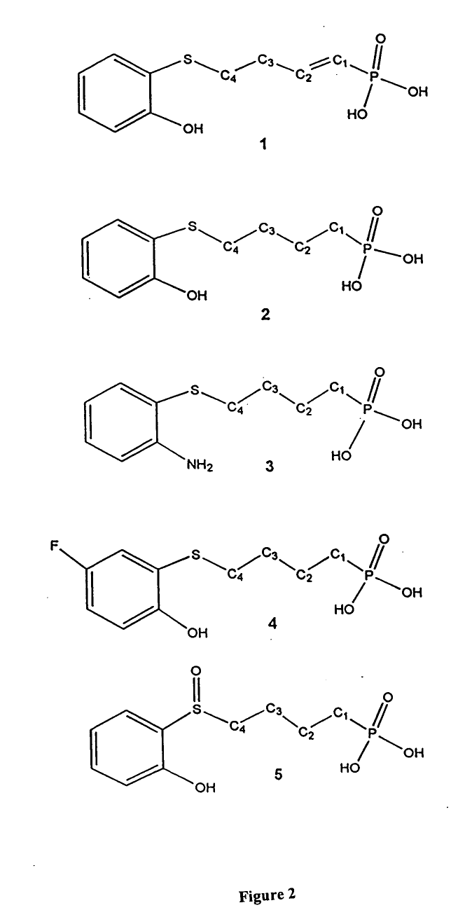

Structural studies on arylthioalkylphosphonate transition state analogues 1-5 (FIG. 2) designed to inhibit the TSα-reaction are described. In order to establish the molecular basis of inhibition by these agents, the crystal structures of the corresponding complexes have been determined at 2.3 Å or better resolution. The information obtained from these experiments has implications for the mechanism of catalysis and studies differences in the mode of binding for inhibitors in an analog series.

Chemicals. The following tryptophan synthase inhibitors were used in this study: 4-(2-hydroxyphenylthio)-1-butenylphosphonic acid, 1:2 salt with isopropylamine (1); 4-(2-hydroxyphenylthio)-butylphosphonic acid, 1:2 salt with diisopropylamine (2); 4-(2-aminophenylthio)-butylphosphonic acid (3); 4-(2-hydroxy-5-fluorophenyl thio)-butylphosphonic acid, 1:1 salt with diisopropylamine (4), and 4-(2-hydroxy phenylsulfinyl)-butylphosphonic acid (5). The compound were prepared are described in the U.S. Pat. No. 5,635,449 to Langevine and Finn. The chemical structures of these inhibitors are shown in FIG. 2.

Crystallization and X-ray Data Collection. The expression and purification of the tryptophan synthase α₂β₂ complex from Salmonella typhimurium was done as described in Miles et al., J. Biol. Chem. 264:6280-6287, 1989. The protein-inhibitor complexes were prepared by mixing the individual components so that the final protein concentration was 5-10 mg/mL and the final inhibitor concentration 10 mM. Crystals of the complexes were grown under conditions (50 mM Bicine, 1 mM Na-EDTA, 0.8-1.5 mM Spermine and 12% PEG 4000 adjusted to pH 7.8 with NaOH) modified from the original protocol to crystallize the unliganded enzyme. The crystals exhibit symmetry of the space group C2 with an αβ pair in the asymmetric unit.

Diffraction data were collected at low temperature (140K) on an Raxis IIC imaging plate system with CuKα X-rays generated from a Rigaku RU-200 rotating anode operating at 50 kV and 100 mA and equipped with a Yale double mirror system. The crystal to detector distance was 100 mm and the oscillation range 1°. Data were processed with DENZO (Otwinowski et al., Methods Enzymol. 276:307-325, 1997) and the CCP4 (Dodson, et al., Methods Enzymol. 277:620-633 1997) suite of programs.

Refinement. The starting model for all five refinements was the coordinate set of a refined model of native TRPS (PDB entry la5s) (Schneider et al., Biochemistry 37:5394-406, 1998) without the cofactor PLP. X-PLOR 3.851 (Brunger, A. T., “E-PLOR 3.85 1 ”, Yale Univ. Press., New Haven, Conn. 1997) was employed for all calculations. The graphics program O (Jones et al., Acta Crystallogr. A47: 110-119, 1991) was used for the display of electron density maps (2F_{obs}−F_{calc} and F_{obs}−F_{calc}, difference syntheses at varying contour levels) and manual rebuilding of atomic models. The R_{free} factor (Brunger, A. T. Nature 355:472-475, 1994) was implemented from the beginning and its value used as a criterion for model improvement during the course of the refinement. After an initial round of rigid body refinement, the model was subjected to a simulated annealing protocol starting at 4000K. At this point, atomic models of the phosphonate inhibitor for each complex and of the common cofactor PLP that were generated and geometrically minimized with InsightII (MSI) were built into the corresponding electron density.

Several rounds of slow cooling protocols with varying weights and starting temperatures, grouped and individual B factor refinement, and manual rebuilding followed. Placement of water molecules was done by selecting the peaks in F_{obs}−F_{calc} difference maps that had heights greater than 4σ and fulfilled hydrogen bonding criteria. A two-parameter bulk-solvent correction (Jiang, et al., J. Mol. Biol. 243:100-115, 1994) was applied and this allowed low resolution (5-30 Å) reflections to be used in the refinement. In the final stages of refinement, the coordinates and B factors of the atomic model were refined by using the conjugate gradient minimization algorithm. Data and refinement statistics are shown in Table 23.

Results

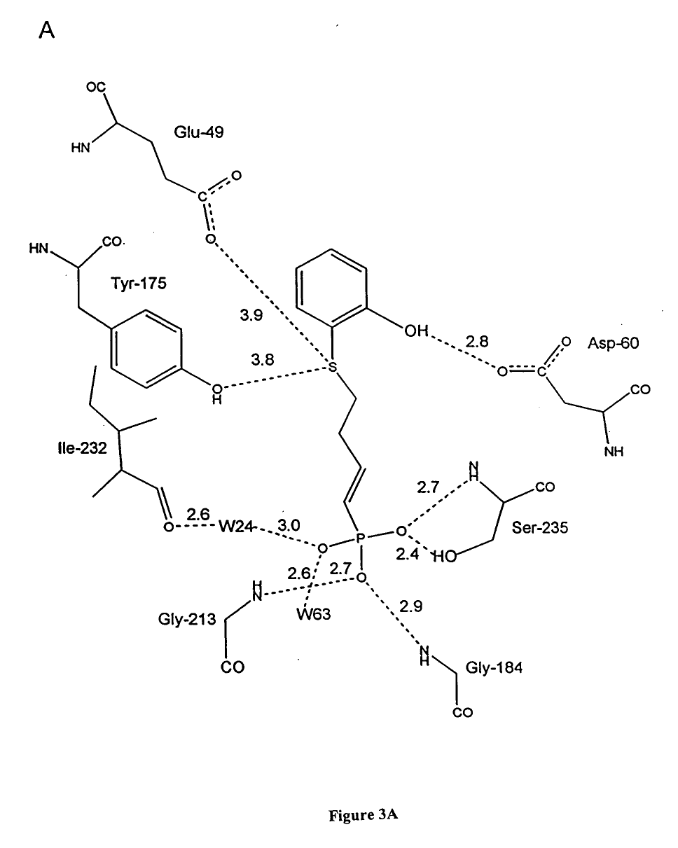

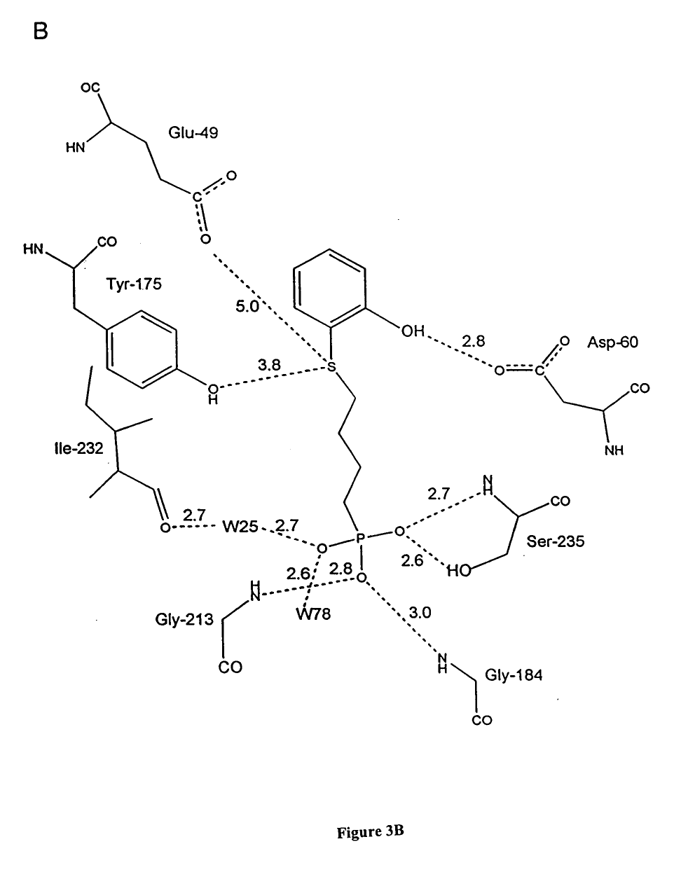

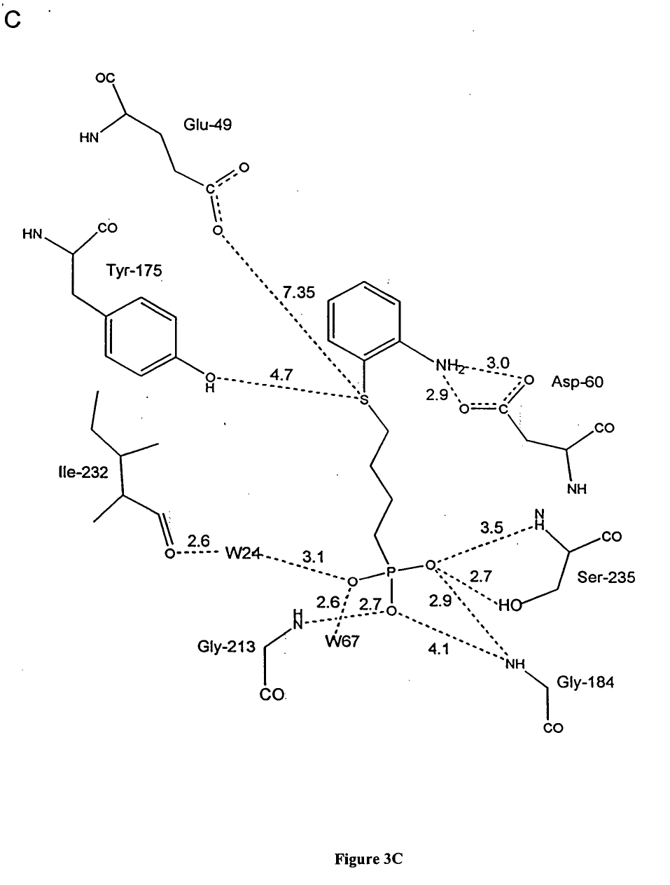

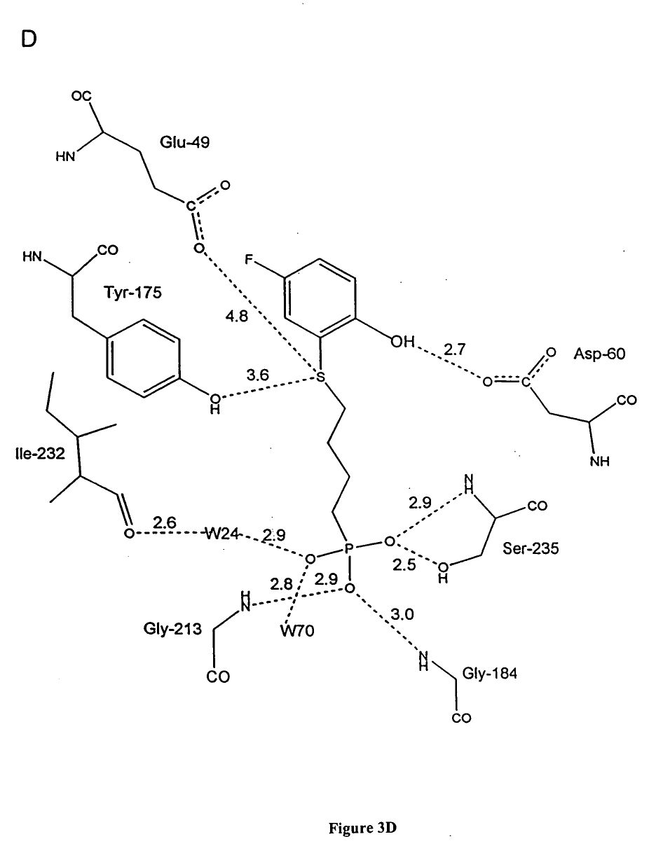

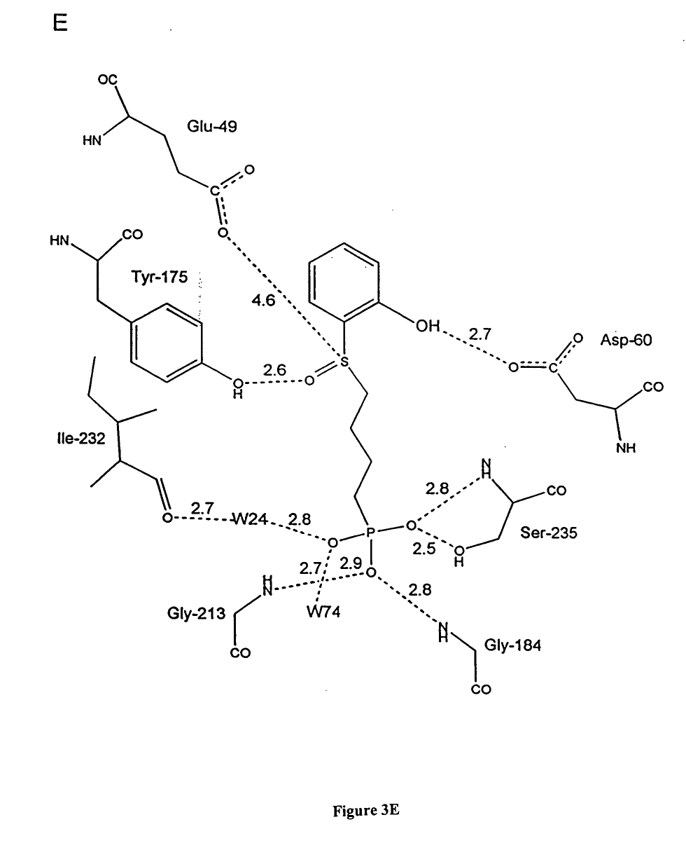

Enzyme-inhibitor interactions. Conventional and simulated annealing-omit electron density maps at 2.3 Å resolution or higher show strong positive features and clearly delineate the phenyl ring, the thiobutyl or thiobutenyl or sulfinylbutyl moieties, and the phosphonate groups of the different inhibitors. As expected, the phosphonate inhibitors bind to the α-reaction binding site. Potential hydrogen bonding interactions and relative distances from active site residues for the different inhibitors, are shown in FIGS. 3A-E. Some interactions are common in all inhibitors, while others are unique and contribute to the different inhibition constants.

The phenyl ring and side chain (thiobutyl, thiobutenyl, or sulfinylbutyl groups) of all inhibitors make contact with a number of hydrophobic residues including Phe-22, Leu-100, Leu127, Phe-212, Leu-232, and the methyl group of Thr-183. This is very similar to the packing of the indole and propyl moieties of IPP, as predicted. The alkylphosphonate portion of the inhibitors extends approximately at a right angle with the phenyl ring, and the phosphonate oxygens form hydrogen bonds with main chain nitrogens of Gly-184, Gly-213, Gly-234 and Ser-235, two water molecules, and the hydroxyl group of Ser-235. The latter interaction (with the hydroxyl of Ser235) appears to be particularly strong in the complexes of TRPS with inhibitors 1, 4 and 5. The o-substituent of the phenyl ring consistently interacts with the carboxylate of the putative catalytic residue Asp-60 (X—O distances range from 2.6-2.8 Å, where X═O or N) (Hodel et al., Acta Crystallogr. A48:851-858, 1992)(Hyde et al., J. Biol. Chem. 263:17857-17871, 1988). The amino group of inhibitor 3 forms two hydrogen bonds with the carboxylate of Asp-60 versus one hydrogen bond for the o-hydroxyl substituted inhibitors. Interestingly, despite the presence of two hydrogen bonds for inhibitor 3, it has a higher IC₅₀ value for enzyme inhibition than the o-hydroxyarylalkyl sulfide inhibitors, which only form one hydrogen bond.

Inhibitor 1 has the highest activity in enzyme inhibitory and herbicidal assays. The structure provides an explanation for its potency. The rigidity introduced by the double bond does not perturb the potential for hydrophobic and van der Waals interactions, yet presumably favors binding due to entropic effects (fewer degrees of freedom are lost upon binding than in the case of a saturated C—C bond). Furthermore, in this conformation, one of the phosphonate oxygens is brought into very close contact with the hydroxyl of Ser-235, forming a strong, possibly low barrier, hydrogen bond (O . . . O interatomic distance refined to 2.4 Å) (Cleland, W. W., Biochemistry 31:317-319, 1992; Cleland et al., Science 264:1887-1890, 1994; Gerlt et al., J. Am. Chem. Soc. 115:11552-11568, 1993; Gerlt et al., Biochemistry 32:11943-11952, 1993). These bonds can have dissociation energies of 12-24 kcal/mol, roughly ten times higher than ordinary hydrogen bonds.

While the o-hydroxyl group of inhibitor 2 forms a strong interaction with the carboxylate of Asp-60 (O—O distance=2.8 Å), the distance of the hydrogen bond is longer than all other inhibitors in this series. The o-amino group of inhibitor 3 makes two hydrogen bonds with the same carboxylate (versus one hydrogen bond for all other inhibitors, which have an o-hydroxy group at this position). The presence of two hydrogen bonds, however, does not increase the affinity of this inhibitor for TRPS relative to the other inhibitors. An explanation of the weaker enzyme inhibitory activity of this compound can be formulated on the basis of superposition with the structure of the TRPS complex with the natural substrate IGP.

Inhibitors 4 and 5 possess two unique atoms that were designed to enhance interactions with TRPS. Surprisingly, the p-fluorine substituent of the ring in inhibitor 4 does not participate in any polar interactions and is in proximity only to the CD1 carbon of Ile-153 (F—C distance=3.1 Å). The sulfoxide oxygen of inhibitor 5 seems to make a strong hydrogen bond with the hydroxyl group of Tyr-175 (O—O distance=2.6 Å). It is interesting to note that the S—O bond in inhibitor 5 refines to a distance of 1.65 Å, much longer than the S—O bond distance in crystalline DMSO (1.47 Å) (Martin et al., “Dimethylsulfoxide”, Wiley Inc., New York, N.Y. 1975). However, the 1.65 Å S—O bond length is close to what is observed in the complex between DMSO and DMSO-reductase (McAlpine et al., J. Mol. Biol. 275:613-23, 1998). In the latter, the interaction of DMSO with molybdenum weakens the S═O double bond, and is consistent with small molecule studies of DMSO ligated to transition metals (Martin et al. 1975). The data represented herein suggest that the S═O . . . H—O-Tyr-175 interaction is strong enough to similarly weaken the S═O double bond character in inhibitor 5. The resonance of the sulfoxide with the phenyl ring, may also contribute to the increase in the length and polarity of this bond.

In the complexes of TRPS with inhibitors 1, 4 and 5, the distance between one of the oxygens of the phosphonate group and the hydroxyl oxygen of Ser-235 has refined to values less than or equal to 2.5 Å implying the involvement of a strong, very short hydrogen bond in the stabilization of the enzyme-inhibitor complexes. The specific distance of this hydrogen bond for each of the inhibitors is as follows: inhibitor 1, 2.4 Å; inhibitor 2, 2.6 Å; inhibitor 3, 2.7 Å; inhibitor 4, 2.5 Å; and inhibitor 5, 2.5 Å. Such very short hydrogen bonds for inhibitors 1, 4, and 5 have been observed in a number of structures of complexes of carboxypeptidase A (Kim et al., Biochemistry 29:5546-5555, 1990; Kim et al., Biochemistry 30:8171-8180 1991), thermolysin (Holden et al., Biochemistry 26:8542-8553, 1987; Tronrud et al., Eur. J. Biochem. 157:261-268, 1986), penicillopepsin (Fraser et al., Biochemistry 31:5201-5214, 1992), HIV-1 protease (Abdel-Meguid et al., Biochemistry 32:7972-7980, 1993), and endothiapepsin (Dealwis, C., Thesis, Birkbeck College, 1993) with a series of phosphonate and phosphinate inhibitors acting as analogues of the transition state for peptide hydrolysis. In all of these complexes one of the oxygens of the phosphorus-containing groups is shown to interact with one of the carboxylate oxygens of either a glutaric acid or an aspartic acid residue. The hydrogen bond distances (O—O distances) range between 2.2 and 2.5 Å. It has been proposed that such short, very strong, low barrier hydrogen bonds (LBBB) can have a significant contribution to enzymic catalysis (Cleland 1992; Frey et al., Science 264:1927-1930). However, the existence of LBHBs within enzyme active sites has recently been disputed based upon theoretical (molecular mechanics and ab initio (quantum mechanical) calculations (Scheiner et al., J. Am. Chem. Soc. 117:6970-6975; Washsel et al, Proc. Natl. Acad. Sci. USA 93:13665-70, 1996) and NNM spectroscopic data (Ash et al., Science 278:1128-32, 1997).

In this example, however, the very short hydrogen bonds are not involved in the catalytic mechanism. There are two other examples of very short hydrogen bonds in enzyme-ligand complexes that bear the closest chemical resemblance to the ones observed in the structures shown herein. In the complex of cytidine deaminase with a TSA inhibitor an interaction occurs between an alcoholic hydroxyl of the inhibitor and a glutamate carboxylate oxygen with a refined O—O interatomic distance of 2.4 Å (Xiang et al., Biochemistry 34:4516-23, 1995). A very similar bond between an aspartate carboxylate group and a hydroxyl of a sugar moiety of a trisaccharide is also found in the structure of a lysozymetrisaccharide complex (Strynadka et al., J. Mol. Biol. 220:401-424). In the case of cytidine deaminase, the hydroxyl group is the predominant feature that distinguishes the transition state from the ground state of the substrate cytidine. In the case of lysozyme, however, this particular hydrogen bond is observed at a site (site B) far from where cleavage of the glycosidic bond of the sugar is proposed to occur (junction of sites D and E). Thus it may simply confer higher affinity of the ligand for the enzyme.

In the case of the enzyme-inhibitor complexes of the invention, consideration of these hydrogen bonds allows one to understand the stronger binding of inhibitor 1 to the α subunit active site. Presumably, the presence of an α-β double bond in conjugation with the phosphonyl group increases the electron density on its oxygen atoms and effectively increases their tendency for formation of strong hydrogen bonds. It is significant to note that in the case of the complex of TRPS with inhibitor 3, the compound that has the weakest activity in the biological and enzyme inhibitory assays, the (P—) O . . . H—O distance is the largest for this series of complexes. This is the first time that such a strong hydrogen bond between a phosphonyl oxygen and an alcoholic hydroxyl oxygen is observed in enzyme-inhibitor complexes.

Comparison of inhibitor and substrate (IGP) binding. The position and interactions of the phosphonate group and the ortho-substituent of the phenyl ring of the inhibitors of the invention are very similar to those of the phosphate group and the indole nitrogen respectively of IGP in the TRPS-IGP complex. However, the actual position and orientation of the phenyl ring and alkyl groups differ significantly from that of the indole ring and glyceryl chain of IGP. Interestingly, in the ortho-hydroxy compounds the phenyl ring seems to be tilted about 30° with respect to the plane of indole whereas the ortho-amino containing inhibitor has its phenyl ring almost parallel to that plane. Since the angle between each ring and the corresponding alkyl chain is roughly the same (90°) in both classes of compounds, the same difference in orientation is observed between the alkyl and the glyceryl chains of the phosphonates and IGP, respectively. The only exception to this is inhibitor 3.

Implications for the mechanism of catalysts. The transition state of the α-reaction is presumed to involve a tetrahedral carbon atom. The C—S—C angle in all of arylthioalkylphosphonate inhibitors in this study varies between 108° and 110°, which is very close to the expected value for a tetrahedrally coordinated atom (109° 28′). This implies that the sulfur atom mimics the putative tetrahedral carbon atom in the transition state. Analysis of the interactions between the inhibitors and the enzyme could therefore be useful in understanding the catalytic mechanism.

The transition state in the (α subunit active site is formed with the assistance of three functional groups: B₁H, B₂, and B₃. Asp-60 and Glu49 have been previously identified as B₂ and B₃, respectively, but the identity of B₁H has remained inconclusive (Rhee et al., J. Biol. Chem. 273:8553-5, 1998). The present structures reinforce the idea that Asp-60 plays a catalytically important role as a base (B₂) that abstracts the proton from the indole nitrogen (—NH—) and facilitates indolenine tautomerization of IGP. In all of the complexes the o-substituent of the phenyl ring, which is in a position equivalent to that of NH— of indole and exerts similar electronic effects on the ring, interacts with the carboxylate of this particular aspartate residue. The inhibitors of the invention do not possess any polar substituent (H-bond donor) on the C-4 of the alkyl group, which is equivalent with the C3′ of the indole of IGP. Such a group could potentially mimic the interactions of the C3′-OH of IGP. Its absence from our inhibitors limits the conclusions that can be drawn from these structures with respect to the nature of the base B₃. However, the recent structure of the complex of a αD60N mutant of TRPS with the natural substrate IGP (Rhee et al. 1998) revealed a strong hydrogen bond between one of the carboxylate oxygens of Glu-49 and the C3′-hydroxyl of IGP (the C3′ of IGP is equivalent to the C-4 of the alkyl group of the present inhibitors), implying that this group can indeed serve as a base that will deprotonate the C3′-hydroxyl during catalysis and facilitate IGP cleavage.

TABLE 23
Clystal Parameters, Data and Refinement Statistics

| | TRPS-1 | TRPS-2 | TRPS-3 | TRPS-4 | TRPS-5 |
|---|---|---|---|---|---|
| CRYSTAL PARAMETERS | | | | | |
| unit cell (a, b, c) (Å) | 183.0, 58.8, 67.7 | 183.8, 60.8, 68.2 | 182.7, 59.3, 67.3 | 184.2, 60.5, 67.8 | 185.1, 60.2, |
| unit cell (β) (deg) | 94.2 | 94.4 | 94.5 | 94.4 | 94.7 |
| data statistics | | | | | |
| resolution (Å) | 44-2.3 | 45.8-2.2 | 42.7-2.3 | 39.4-2.3 | 39.4-2.0 |
| no of collected reflections | 245,222 | 223,028 | 110,360 | 95,281 | 258,965 |
| no of unique reflections | 29,830 | 35,625 | 30,288 | 31,780 | 53,052 |
| compl. (total/high) (%) | 92.6/85.2 | 93.5/87.2 | 90.6/70.8 | 95.2/79.6 | 95.4/87.8 |
| Rₘ (total/high) (%) | 7.6/16.1 | 7.8/18.3 | 11.7/27.6 | 7.3/17.3 | 5.5/16.2 |
| <I/σ(I)> (total/high) | 12.9/3.9 | 7.4/2.9 | 8.0/4.1 | 11.2/4.5 | 10.8/2.5 |
| refinement statistics | | | | | |
| resolution range (Å) | 30-2.3 | 30-2.2 | 30-2.3 | 30-2.3 | 30-2.0 |
| no of reflections with F > 2σ(F) | 29,402 | 35,371 | 29,619 | 31,553 | 52,755 |
| no. of protein atoms | 4979 | 4979 | 4979 | 4979 | 4979 |
| no. of waters | 167 | 169 | 161 | 191 | 190 |
| no. of other atoms | 31 | 31 | 31 | 31 | 31 |
| R_{work}, R_{free} (%) | 28.1/23.0 | 28-2/21.6 | 28.4/24.0 | 26.8/23.0 | 27.3/23.0 |
| rmsd for bonds/angles (Å/deg) | 0.009/1.82 | 0.013/2.05 | 0.006/1.66 | 0.008/2.44 | 0.009/2.76 |
| disallowed (Φ, Ψ) | Phe212 | | | Phe212 | Phe212 |
| <B> (mc/sc/wat) (Å) | 15.4/20.3/21.6 | 12.1/15.9/20.9 | 17.1/22.6/19.7 | 14.3/18.7/16.59 | 16.8/22.4/22.8 |
| <esd> (Å) | 0.31 | 0.22 | 0.33 | 0.28 | 0.27 |

Completeness, Rₘ, and <I/σ(I)> are given for all data and for data in the highest resolution shell.
Rm = Σ|IαI>|/Σ/.
No unambiguous electron density was found for the following residues in the atomic model: α1, α188-193, α268, β1, β394-397.
Other atoms represent PLP in all cases and the corresponding phosphonate inhibitor in each complex.
Mean thermal B factors are given for main chain (mc) and side chain (sc) protein atoms and water molecules (wat).
<esd> is the mean coordinate error estimated by the SIGMAA method.

### Example 19

Computational searches in chemical databases to find novel compounds or compound fragments to improve inhibitor binding or herbicidal activity can result in novel synthetic ideas. In the following an example is given for the use of the Ludi program (MSI) for this purpose. LUDI, by design, is a “idea generation” tool. It requires someone skilled in the art to analyze the fragment hits that it generates. It is shown here that such approaches allow the synthetic chemist to find sites for modification of initial leads to rapidly improve the desired compound profile.

A crystal structure of TS, preferably one with a known inhibitor is used as a template. The inhibitor is, however, ignored within the computational approach described here, by removing it from the assembly of the protein and keeping it as copy within separate entity for display purposes. (The whole procedure was performed using the interactive graphics package Insight II (MSI). However, the setup listed below, can be used in a stand alone fashion to run the LUDI program).

The Biosym Fragment Library (MSI) (1996 version) was used with the parameters given in Table 24.

TABLE 24

| | |
|---|---|
| CUTOFF | 5.000000 |
| RMSMAX | 0.600000 |
| PESEL | 2.000000 |
| VDWCUT | 3.000000 |
| ESCUT | 2.500000 |
| ANGMAX | 0.000000 |
| IOUT | 0 |
| IELEC | 1 |
| IDENSL | 25 |
| IDENSP | 25 |
| IFLAGV | 0 |
| ILINK | 0 |
| IANALG | 0 |
| IBIFUR | 0 |
| ICONMI | 0 |
| WLINK | 1.000000 |
| WLIPO | 1.000000 |
| WHBOND | 1.000000 |
| INEWSC | 0 |
| IMINSC | 0 |
| NHITS | 940 (set to |
| | number of fragments |
| | in DB) |
| IBINRD | 0 |
| ITARGT | 0 |
| IBURID | 0 |
| ICAVMX | 0 |
| INVERT | 0 |
| IROT | 1 |

The center of the search is set to positions close to the inhibitor ring system, the center of the linker, or the approximate location of the phosphate/phosphonate group, or any other site, that is sought to be filled with novel fragments. The program calculates so-called interaction sites within the cutoff radius of the center of search, e.g., hydrogen bonding sites, van-der-Waals surfaces etc. The fragments from the library are then placed within this model of the binding site and, after optimization of the placement, a score is calculated that describes the match of complementary features. High scoring fragments are saved for later, interactive analysis. After completion of the run, a person skilled in the art can analyze the hits, using the interactive graphic capabilities as implemented in the program Insight II (MSI). The fragments usually only represent a part of the inhibitor molecule since the fragments in the database are too small to represent highly specific and tight binding compounds by themselves.

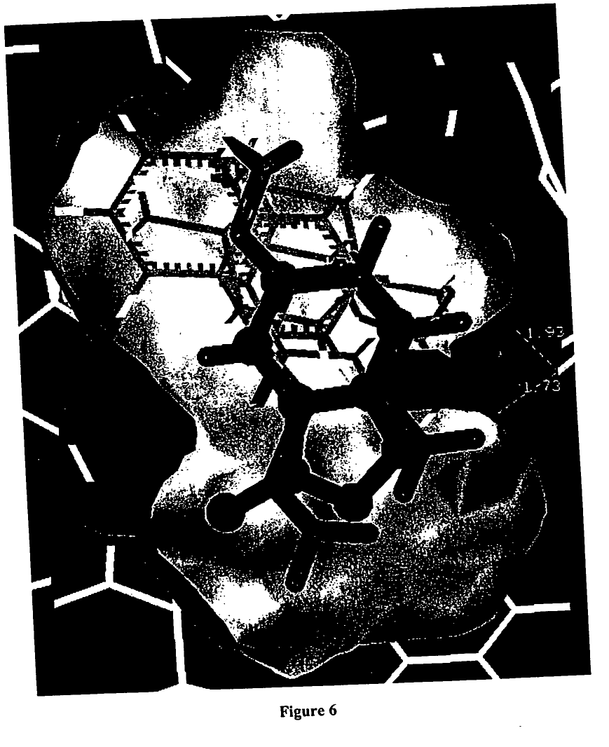

The first result of such a search is a better knowledge of sites that are not fully accessed by the inhibitor. For example, FIG. 6 demonstrates that many fragments are found that extend into a part of the substrate binding site that is not filled by the IPP inhibitor. Modifications, such as the addition of a methoxy group or a halogen atom to the C5 position of the indole (e.g., 5-flouro-indole-propanol-3-phosphonic acid) residue or the C5 position {4-aryl-thiobutyl}-phosphonic acid derivatives.

The fragments found are further evaluated with respect to synthetic feasibility, i.e. the possibility of synthesizing the fragments in the context of a larger inhibitor. E.g. Fragments fitted into the indolyl residue binding pocket need to be evaluated for their potential to be connected synthetically to the thioaryl-liker.

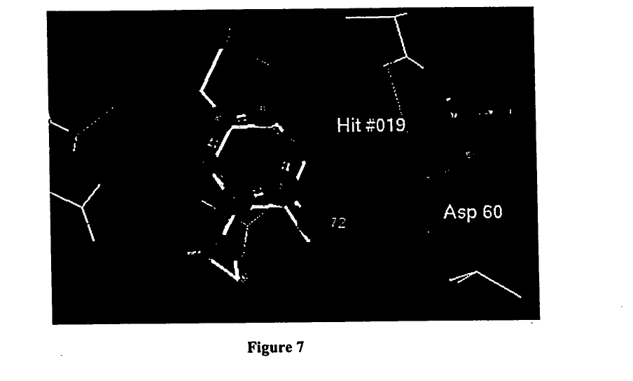

There are other secondary considerations, too that will be influencing the decision of how to use the computationally suggested fragments. Many fragments are found for the linker region that from hydrogen bonds with the enzyme. It is however understood by someone skilled in the art that enthalpy gains from those interactions implemented in the score function of the LUDI program are mostly not reflected in a corresponding true reduction of free energy for inhibitor binding due to loss of hydration of the inhibitor in solution and entropic effects. However, such changes can be considered when implementing synthetic changes for other purposes. For example, linker variations of this amide bonds have been studied that would enable hydrogen bonding interactions in the linker region and, at the same time introduce “metabolic handles” to reduce the lifetime of the inhibitors in crop plants. Furthermore, novel synthetic strategies can be implemented. For example, many fragments indicate that the indole NH group can be replaced by an OH group. In fact, the compounds with a OH group are between the best herbicides of the series. FIG. 7 shows a fragment hit (Hit 19) for which an overlay between the amino group of {4-[2-amino-5-methoxy-phenyl)thio]butyl}-phosphonic acid is shown.

### Example 20

### Homology Modeling

The effective design of inhibitors, the understanding of binding inhibitors on the molecular level and the binding specificity of inhibitors in various crops and weeds relies at least in part on the knowledge of detailed structural models of the TS enzyme's active site.

Homology modeling approaches are an effective way of generating highly accurate structures if structural information about closely related proteins structures are available.

This example describes the generation of a protein model for the maize αTS subunit. Similarly, the whole enzyme can be generated and models of other species can also be obtained by similar steps.

The amino acid sequence for Maize αTS was obtained from the public databank (Accession: pir:S56665). Using the program Quanta (MSI) the sequence of the Maize enzyme was aligned using default settings for the alignment steps to the sequence of the αTS sequences of several known αTS structures (Accession: pdb: trs,pdb:tys, and the complexes TS/{4-[2-amino-5-methoxy-phenyl)thio]butyl}-phosphonic acid and TS/{4-[2-amino-5-chlorophenyl)thio]butyl}-phosphonic acid.

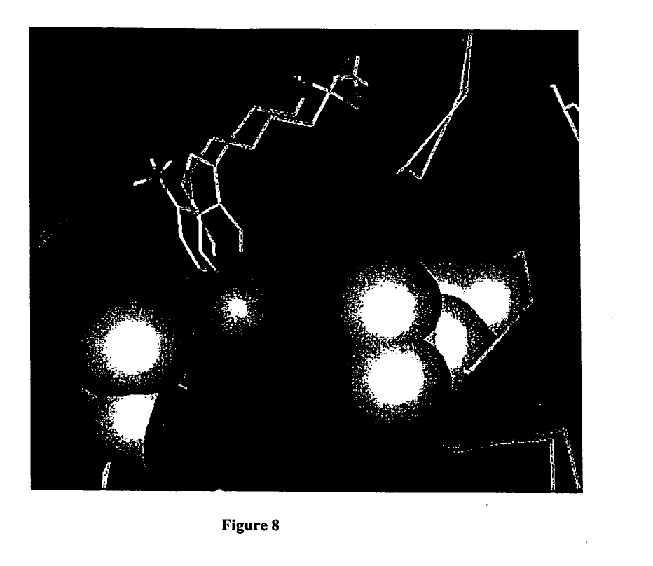

Using the program “modeler” (MSI) in its highest refinement mode, 50 models for the maize enzyme are generated, and scored. The 5 best scoring models are then subjected to a detailed analysis using the program procheck (Laskowski et al., J. Appl. Cryst., 26:283-291). This allows identification of regions in the model that are of low quality and require additional refinement. In this case, the structure proved to be of very good quality and no further additional refinement was necessary. The inhibitor molecules were placed into the model by first placing them into the protein model in a position analog to the one in the template structure. The orientation of the inhibitor and surrounding amino acids is then optimized using appropriate potential energy function based methods. The analysis of the binding site of the maize enzyme revealed that there are only very few changes in the composition of the amino acids contributing to the substrate/inhibitor binding in the αTS active site. A strongly conserved site between such evolutionary distant organisms indicates that careful mutations of amino acids in a crop species could prove very beneficial since there might not be a large amount of natural resistance to novel herbicides. To select potential mutation sites, amino acids directly involved in binding the inhibitor are first selected. For example, sites particularly favored for mutations are those (1) that are close to the location of the entrance of the binding site and (2) that are not in direct contact to the substrate but have close contacts to several of the inhibitors (described herein). Those residues are of high interest for mutations to generate herbicide resistance. Such sites would, e.g., be αAla 129 or αLeu 153 (See FIG. 8). The table below lists corresponding sites in the Salmonella and Maize enzyme that are directly involved in substrate/inhibitor binding.

TABLE 25
Corresponding sites in TS from Salmonella and maize.

| S. th. | Z. Maize |
|---|---|
| PHE 22 | TYR 107 |
| GLU 49 | GLU134 |
| GLY 51 | GLY 136 |
| ALA 59 | ILE 144 |
| ASP 60 | ASP 145 |
| GLY 61 | GLY 146 |
| THR 63 | ILE 148 |
| ILE 64 | ILE 149 |
| ASN 68 | VAL 153 |
| LEU 100 | LEU 184 |
| TYR 102 | TYR 186 |
| LEU 127 | ILE 207 |
| ALA 129 | PRO 209 |
| ILE 153 | LEU 233 |
| TYR 173 | PHE 253 |
| TYR 175 | LEU 256 |
| LEU 177 | VAL 257 |
| ARG 179 | VAL 259 |
| VAL 182 | VAL 262 |
| THR 183 | THR 263 |
| GLY 184 | GLY 264 |
| ALA 185 | PRO 265 |
| GLU 186 | ARG 266 |
| ASN 187 | ALA 267 |
| GLY 211 | GLY 291 |
| PHE 212 | PHE 292 |
| GLY 213 | GLY 293 |
| ILE 214 | ILE 294 |
| ILE 232 | ILE 312 |
| SER 233 | ILE 313 |
| GLY 234 | GLY 314 |
| SER 235 | SER 315 |
| ALA 236 | ALA 316 |
| ILE 237 | MET 317 |
| VAL 238 | VAL 318 |
| PHE 22 | TYR 107 |
| GLU 49 | GLU 134 |
| GLY 51 | GLY 136 |
| ALA 59 | ILE 144 |
| ASP 60 | ASP 145 |
| GLY 61 | GLY 146 |
| THR 63 | ILE 148 |
| ILE 64 | ILE 149 |
| ASN 68 | VAL 153 |
| LEU 100 | LEU 184 |
| TYR 102 | TYR 186 |
| LEU 127 | ILE 207 |
| ALA 129 | PRO 209 |
| ILE 153 | LEU 233 |
| TYR 173 | PHE 253 |
| TYR 175 | LEU 256 |
| LEU 177 | VAL 257 |
| ARG 179 | VAL 259 |
| VAL 182 | VAL 262 |
| THR 183 | THR 263 |
| GLY 184 | GLY 264 |
| ALA 185 | PRO 265 |
| GLU 186 | ARG 266 |
| ASN 187 | ALA 267 |
| GLY 211 | GLY 291 |
| PHE 212 | PHE 292 |
| GLY 213 | GLY 293 |
| ILE 214 | ILE 294 |
| ILE 232 | ILE 312 |
| SER 233 | ILE 313 |
| GLY 234 | GLY 314 |
| SER 235 | SER 315 |
| ALA 236 | ALA 316 |
| ILE 237 | MET 317 |
| VAL 238 | VAL 318 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all sizes and all molecular weight or molecular mass values are approximate, and are provided for description.

Patents, patent applications, procedures, and publications cited throughout this application are incorporated herein by reference in their entireties.

1-39. (canceled).

40. A method for controlling undesired plant growth comprising administering to a plant an herbicidal effective amount of a compound that inhibits tryptophan synthase activity by binding to an active site of a tryptophan synthase, wherein the active site is selected from the group consisting of an alpha subunit of said tryptophan synthase, a beta subunit of said tryptophan synthase, the hydrophobic tunnel connecting the alpha subunit with the beta subunit of said tryptophan synthase, and a combination thereof.

41. The method of claim 40, wherein the undesired plant growth comprises unwanted weed growth.

42. The method of claim 40, wherein the herbicidal effective amount is less than 20 μM.

43. The method of claim 40, wherein the herbicidal effective amount is less than 10 μM.

44. The method of claim 40, wherein the herbicidal effective amount is between 500 nM and 10 μM.

45. The method of claim 40, wherein the herbicidal effective amount is that amount which is sufficient to kill said undesired plant growth.

46. The method of claim 40, wherein the plant is a crop plant.

47. The method of claim 40, wherein the compound comprises an ariel sulfide phosphate.

48. The method of claim 40, wherein the compound comprises an ester or salt form of the compound.

49. The method of claim 40, wherein the compound is dispersed in an aqueous carrier.

50. The method of claim 40, wherein the compound is selected by the steps comprising:
testing the compound in an in vitro assay comprising tryptophan synthase or a subunit thereof, wherein said in vitro assay can detect the activity of said tryptophan synthase or the subunit thereof; and
determining whether tryptophan synthase activity is inhibited by said compound.

51. A method for controlling undesired plant growth comprising administering to a plant an herbicide that is an inhibitor of plant tryptophan synthase.

52. The method of claim 51, wherein the herbicide is administered in an effective amount that inhibits tryptophan synthase activity.

53. The method of claim 52, wherein the herbicide binds to an active site of said plant tryptophan synthase.

54. The method of claim 53, wherein the active site is selected from the group consisting of an alpha subunit of said plant tryptophan synthase, a beta subunit of said plant tryptophan synthase, the hydrophobic tunnel connecting the alpha subunit with the beta subunit of said plant tryptophan synthase, and a combination thereof.

55. The method of claim 51, wherein the undesired plant growth comprises the undesired growth of one or more weeds.

56. The method of claim 51, wherein the herbicide comprises arylthioalkyl- and arylthioalkenylphosphonic acid dialkyl esters.

57. The method of claim 51, wherein the herbicide comprises a compound selected from the group consisting of phosphonic acid,{4-[(2-amino-5-bromophenyl)thio]butyl}; phosphonic acid,{4-[(2-amino-5-bromophenyl)thio]butyl}-diethyl ester; phosphonic acid, {4-[(2-amino-5-chlorophenyl)thio]butyl}; phosphonic acid, {4-[(2-amino-5-chlorophenyl)thio]butyl}-diethyl ester; phosphonic acid, {4-[(o-hydroxphenyl)thio]butyl}; phosphonic acid, {o-hydroxyphenyl)thio]butyl}-diethyl ester; and combinations thereof.

58. The method of claim 57, wherein the compound is administered at a concentration of less than 1000 μM.

59. The method of claim 57, wherein the compound is administered at a concentration of between 1000 μM and 7.8 μM.

60. The method of claim 57, wherein the compound is administered at a concentration of between 500 μM and 16 μM.


SEQUENCE LISTING (8 sequences)

SEQ ID NO: 1 - DNA, 24 bp, Artificial Sequence
  Description of Artificial Sequence Primer
gggttggatccatggcgattgctt

SEQ ID NO: 2 - DNA, 24 bp, Artificial Sequence
  Description of Artificial Sequence Primer
gattcggatccatggcttctctct

SEQ ID NO: 3 - DNA, 25 bp, Artificial Sequence
  Description of Artificial Sequence Primer
aacaaggatccgtagcattcatacc

SEQ ID NO: 4 - DNA, 25 bp, Artificial Sequence
  Description of Artificial Sequence Primer
tatcgatttcgaacccgggtaccga

SEQ ID NO: 5 - DNA, 24 bp, Artificial Sequence
  Description of Artificial Sequence Primer
aacagggatccgcagcctcaggca

SEQ ID NO: 6 - DNA, 25 bp, Artificial Sequence
  Description of Artificial Sequence Primer
gtttctcgaattcaaacatcaagat

SEQ ID NO: 7 - DNA, 25 bp, Artificial Sequence
  Description of Artificial Sequence Primer
tcgtctggatccaagtcatcatcct

SEQ ID NO: 8 - DNA, 22 bp, Artificial Sequence
  Description of Artificial Sequence Primer
acccggatccttcggtcggttt